(19) United States
(12) Patent Application Publication
(10) Pub. No.: US 2009/0253651 A1
(43) Pub. Date: Oct. 8, 2009

(54) DRUG DELIVERY SYSTEM BASED ON REGIOSELECTIVELY AMIDATED HYALURONIC ACID

(75) Inventors: Stefano Norbedo, Trieste (IT); Susanna Bosi, Monfalcone (IT); Massimo Bergamin, Trieste (IT); Riaz Ahmed Khan, Berkshire (GB); Erminio Murano, Trieste (IT); Francesca Dinon, Trieste (IT)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: EURAND PHARMACEUTICALS LTD., Bray, County Wicklow (IE)

(21) Appl. No.: 12/375,379

(22) PCT Filed: Jul. 27, 2007
(86) PCT No.: PCT/EP07/57772

§ 371 Date: Jan. 27, 2009

(30) Foreign Application Priority Data
Jul. 28, 2006 (IE) 20060565

(51) Int. Cl. A61K 31/728; C07H 5/06; C07K 2/00

(52) U.S. Cl. 514/54; 536/55.1; 530/322

(57) ABSTRACT

New drug delivery systems (DDS) are described containing hyaluronic acid and a therapeutic agent, wherein the therapeutic agent is linked, directly or via a linker, to 6-aminohyaluronic acid and where the linkage of the drug or linker with 6-aminohyaluronic acid is realised by an amide bond. Preferred therapeutic agents for use in the present DDS are anti-inflammatory, antibiotic, antitumor drugs. Preferred linkers are: succinic acid, succinic acid linked to aminoacids, succinic acid linked to peptides. The DDS are stable and free of undesired reaction by-products and impurities, and show a high level of pharmacological efficacy.

### FIELD OF THE INVENTION

The present invention relates to a novel drug delivery system (DDS) wherein hyaluronic acid is linked to a therapeutic agent by amide linkage at a specific position in the polymer, either directly or through a linker.

### PRIOR ART

Amongst the problems encountered in different types of therapies, such as cancer therapy are: (a) small molecule (anticancer drugs) are mainly hydrophobic in nature hence have poor water solubility and consequently their biological properties are impaired and (b) insufficient selectivity for specific tissues or cells. For example, camptothecin (CPT) is a water insoluble, optically active alkaloid obtained from Camptotheca acuminata tree. 20(S)-Camptothecin and its analogues are cytotoxic agents that are thought to act by stabilising a topoisomerase I-induced single strand in the phosphodiester backbone of DNA, thereby preventing relegation. This leads to the production of a double strand DNA break during replication, which results in apoptosis, if not repaired. 20(S)-Camptothecins exhibit excellent antitumour activity against human cancer cell lines and in vivo animal xenografts. In addition to its water insolubility, its pharmacologically important lactone ring is unstable in human plasma where it is present mainly as its open hydroxy-acid form, which is captured by albumin, thus inactivating the drug. The primary limitations of camptothecins are the formation of a labile drug target complex and instability of the lactone ring. On the basis of the reversibility of the ternary complex and formation of lethal lesions during DNA replication, optimal cytotoxic effects are expected with prolonged exposure to the drug. Several camptothecin (CPT) analogues have undergone clinical development but despite their promising clinical role, the over all therapeutic impact of available CPT analogues has been modest; many approaches to optimise their therapeutic indices are being evaluated. Researchers tried to solve the problems by preparing new compounds. Topotecan and irinotecan are synthetic analogues designed to facilitate parenteral administration of the active lactone form of the compound by introducing functional groups to enhance solubility. They are now well-established components in the chemotherapeutic management of several neoplasms. Topotecan has good activity in patients treated previously with ovarian and small cell lung cancer and is currently approved for use in the United States as second-line therapy in these diseases. Irinotecan is a prodrug that undergoes enzymatic conversion to the biologically active metabolite 7-ethyl-10-hydroxy-camptothecin (SN38). It is presently the treatment of choice when used in combination with fluoropyrimidines as first-line therapy for patients with advanced colorectal cancer or as a single agent after failure of 5-fluorouracil-based chemotherapy. Several additional camptothecin analogues are in various stages of clinical development, including 9-aminocamptothecin, 9-nitrocamptothecin,7-(4-methylpiperazinomethylene)-10,11-ethylenedioxy-20(S)-camptothecin, exatecan mesylate, and karenitecin. These compounds however present some disadvantages such as they are not suitable for targeting to specific tissues or cell receptors.

In the attempt to optimize drug efficacy, possible strategies may involve chemical modifications of molecular structure. One strategy is to covalently bind the drug to a water soluble polymeric material. A number of polymers have been used to conjugate hydrophobic drug molecules, including camptothecins, such as poly(L-glutamic acid)-paclitaxe; polyethylene glycol (PEG)-camptothecin (Rowinsky E K et al., J. Clinical Oncology, Vol. 21, No. 1 (2003) 148-157); poly(L-glutamic acid)-camptothecins (Singer J W et al., Ann. N Y Acad. Sci. 922 (2000) 136-150); poly(N-hydroxypropylmethylacrylamide)-HA-doxorubicin, carboxymethyl dextran-camptothecin (CMD Clinical Cancer Research, 11, 1650-1657, 2005), cyclodextrin and CPT (Cheng J, Bioconjugate Chem. 2003, 14, 1007-1017). Such strategies however do not allow to have a targeting to the desired sites; in some cases the polymer does not recognise any targets at all either because of its very chemical nature or because it has lost its original/native targeting capability, due to the chemical modification. In fact, the functional and biological properties of the native polymer may be easily lost when the chemical modification involves groups that are essential for their maintenance.

Paclitaxel (TXL) is an antileukemic and antitumour agent. It was first isolated from the bark of the Pacific yew tree, Taxus bravifolia, has shown high activity against a wide range of tumours and has been clinically used in the treatment of Metastatic breast cancer, refractary ovarian cancer and several other malignancies. TXL is a highly hydrophobic drug with very low solubility in water in its native form. The sustained infusions of TXL have exhibited greater clinical efficacy than bolus injections or more rapid infusion rates. In order to overcome the solubility problem and to enhance its clinical efficacy by sustained infusion of the drug, TXL has been conjugated with polyethylene glycol (PEG) by introducing an accessible ketone group through esterification of the parent drug with acetylbenzoyl chloride, followed by reaction with a series of maleimide containing acylhydrazides (Rodrigues P C A et al., Bioorg. Med. Chem. Lett., 13, 2003, 355). TXL has also been conjugated to hyaluronic acid (also indicated in this application as HA) in order to overcome the solubility problem and to target the tumour cells. The synthetic strategy involved the conjugation of the drug at the carboxylic group of the glucuronic acid residue of HA involving sequential treatment of TXL with succinic anhydride, activation of the TXL-hemisuccinate, functionalisation of the carboxyl group of HA with adipic dihidrazide, and then reacting the two intermediates afforded the HA-Taxol conjugate. The conjugate exhibited selective toxicity toward the human cancer cell lines (breast, colon and ovarian) that are known to express hyaluronic acid receptors; no toxicity was noted against a mouse fibroblast cell line at the same concentrations (Luo, Y., and Prestwich, G. D., Bioconjugate Chem., 10 (1999) 755-763). However these derivatives are indiscriminately substituted at different positions of the polymer thus losing the native chemical regularity.

Poly(L-glutamic acid), polyethylenglycol (PEG) and carboxymethyldextran (CMD) lack in bioactivity and targeting capabilities; while native HA has shown advantages over the other polymers because of its capability to target the drug to the diseased site. Anti-cancer polymeric drugs can traverse through the cancer site either by enhanced permeability and retention (EPR) module, a passive mechanism, or by active targeting using specific interactions between receptors on the cell surface. HA can not only operate through the EPR module, but also have a number of recognised cell receptors in the body and it may interact with other structures such as in particular proteoglycans. Among the different receptors, the CD44 may be quoted. Studies of interaction between hyaluronic acid and proteic receptors (e.g. CD44) have revealed that the binding occurs between the negatively charged carboxyl groups of the hyaluronic acid and the domains of positively charged basic aminoacid acid of the protein (J. Cell. Biol. vol 22, 1993, 257-264). Free carboxyl groups are also required for the interaction of hyaluronic acid with proteoglycans (Biochem. J. 167, 711, 1977). So, integrity of carboxylic groups and hydroxyl groups of hyaluronic acid are very important for recognition and binding with cell structures; a partial substitution of these groups would not allow an effective binding with these proteic macromolecules. These free carboxylic and hydroxylic groups are also important for the formation of proper polymeric conformation. With regards to the receptor CD44, it is well known that many tumor types overexpress it. Endocytosis of derivatised HA has been shown in cell lines expressing CD44 HA receptor. The fluorescent labelled HA-Taxol conjugate has been shown to be selectively toxic towards human cancer cell lines which were known to overexpress HA receptors. The presence of liver receptors for HA (HARLEC) suggests that it can be used as a carrier molecule to target a drug to the liver tissue. HA has been demonstrated for liver metastases from a colon adenocarcinoma in mice.

It has been already described the preparation of a derivative of hyaluronic acid substituted on position C6 by drugs belonging to methotrexate family (WO01168105). These DDS are characterised by the presence of an ester group between the hyaluronic acid and the drug which has specific characteristic profile of drug release and stability at different biological ambients.

### DESCRIPTION OF THE FIGURES

FIG. 1: Synthesis of 6-NH₂-HA

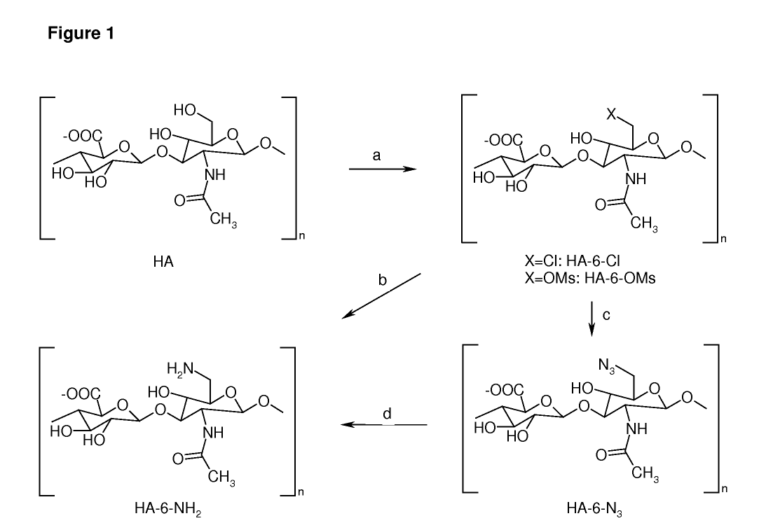

HA→6-Cl-HA; HA-6→Ms→6-NH₂-HA

Conditions: a) X═Cl: MsCl, DMF, A; X═OMs: MsCl, DMF, DIEA, −10° C.; b) aq. NH₄OH, Δ; c) X═Cl: NaN₃, DMSO, 18-crown-6, Δ; X═OMs: NaN₃, water, Δ; d) NH₄COOH, water, Pd/C, RT.

FIG. 2: Synthesis of HA-6-NH—CO—(CH₂)₂—CO-20-O-CPT.

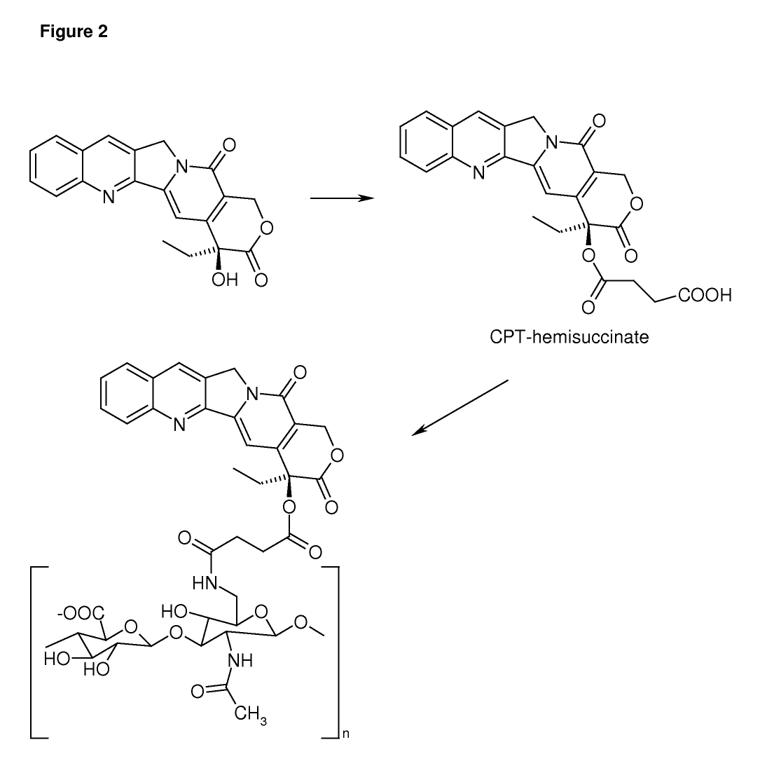

CPT CPT-Hemisuccinate HA-6-NH-Succinate-20-0-CPT

FIG. 3: Synthesis of HA-6-NH—CO—(CH₂)₂—CO-2′-O-TXL.

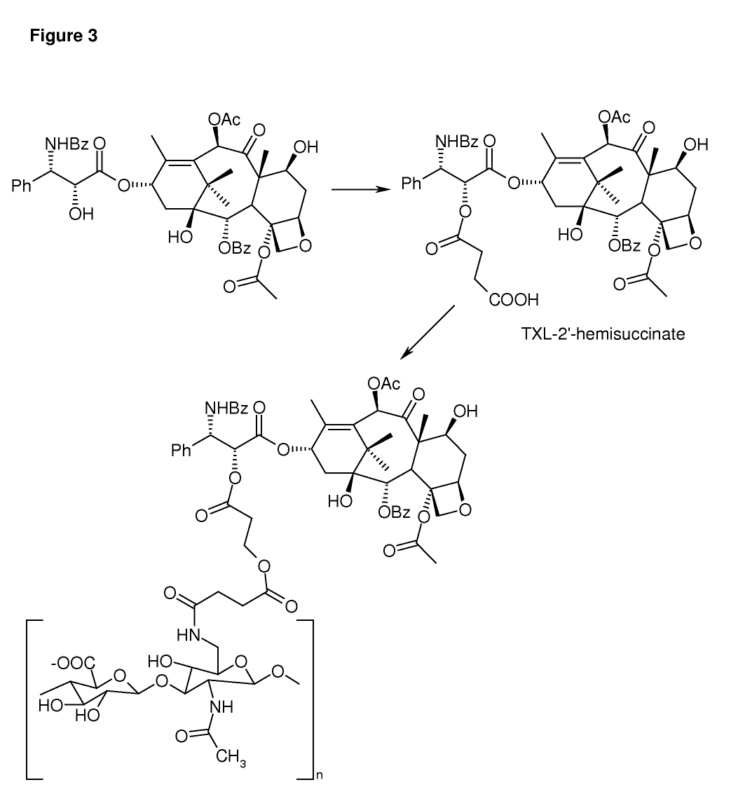

TXL→TXL-Hemisuccinate HA-6-NH-Succinate-2′-O-TXL

FIG. 4: HA-6-NH—CO—(CH₂)₂—CO-Gly-20-O-CPT.

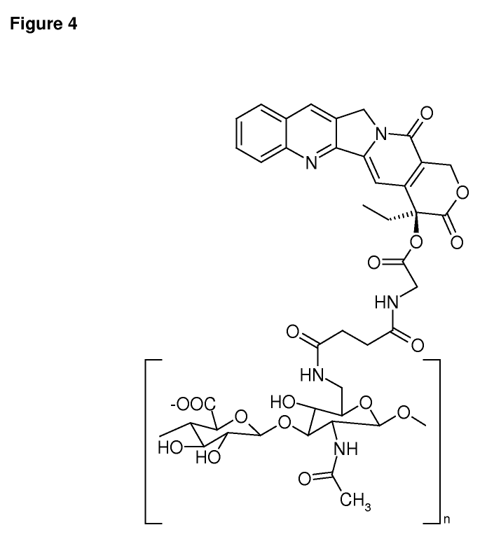

FIG. 5: HA-6-NH—CO-gly-CO—(CH₂)₂—CO-20-O-CPT

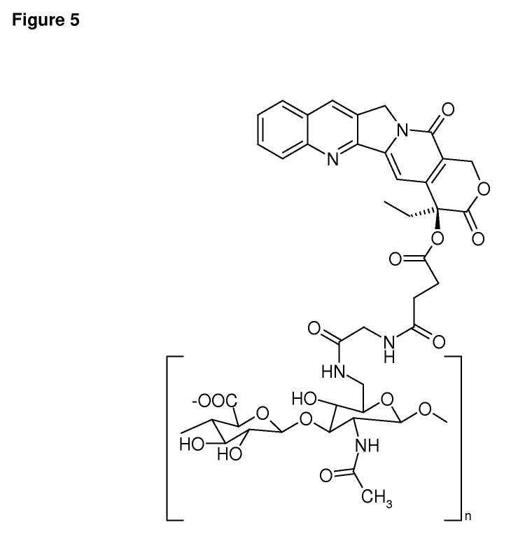

FIG. 6: HA-6-NH—CO-MTX

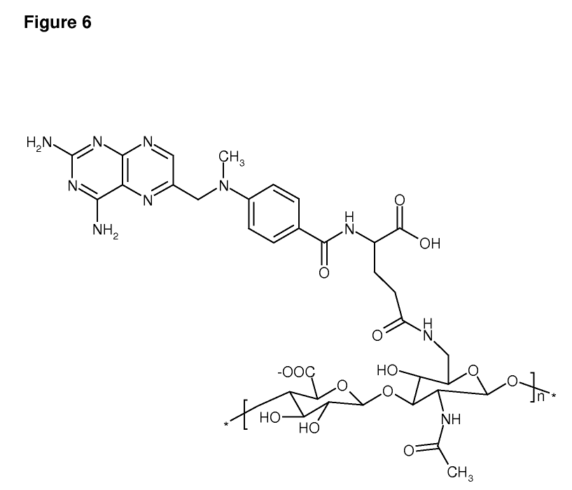

FIG. 7: HA-6-NH—CO-IBP

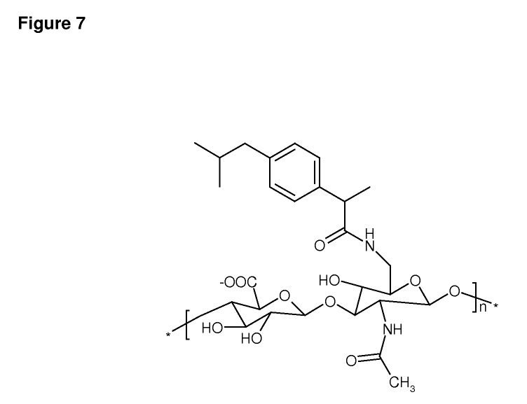

### DETAILED DESCRIPTION OF THE INVENTION

Object of the present invention are DDS containing hyaluronic acid and a therapeutic agent, characterised in that the therapeutic agent is linked, directly or via a linker, to a hyaluronic acid derivative bearing an amino group in the C6 position of the N-acetyl-D-glucosamine residue; this amino group replaces the hydroxy group naturally present at this position in HA. This derivative is herein referred as “6-aminohyaluronic acid” or “6-NH₂-HA”, or “HA-6-NH₂” for brevity; equivalent terminology is used herein for the other 6-derivatised hyaluronic acids, where NH₂ is replaced by the relevant substituent. The linkage of 6-NH₂-HA with the therapeutic agent (or with the linker) is realised by an amide bond involving, on one side, the 6-amino group of 6-NH₂-HA group and, on the other side, a suitable COOH group present on the therapeutic agent (or on the linker). When a linker is present, the therapeutic agent is further linked to the linker by covalent binding.

The degree of substitution, i.e. the percent of 6-NH₂ groups HA involved in the amino linkage with the therapeutic agent or linker, is variable depending on the amount of therapeutic agent/linker used in the amide formation reaction. The degree of substitution is thus easily controlled, resulting in DDS having different levels of drug loading, useful for different therapeutic purposes.

The “hyaluronic acid” (or “HA”) contained in the present DDS is a polymer composed of a disaccharidic repeating unit, consisting of D-glucuronic acid and 2-acetamido-2-deoxy-D-glucose (N-acetyl-D-glucosamine) bound by β(1→3) glycosidic linkage; the D-glucuronic acid residue may either be in the acid form or in the form of a salt. Each repeating unit is bound to the next one by a β(1→4) glycosidic linkage that forms a linear polymer. The hyaluronic acid has preferably an average molecular weight comprised from 10,000 to 1 million and more preferably from 10,000 to 500,000. The term “hyaluronic acid” or “HA” encompasses both the free acid and its salified form with e.g. alkaline metals (preferably Na or K), earth-alkaline metals (preferably Ca or Mg), transition metals (preferably Cu, Zn, Ag, Au, Co, Ag). The terms “hyaluronic acid”/“HA” also include derivatives thereof wherein one or more secondary hydroxyl groups are derivatised to form e.g. groups selected from: —OR, —OCOR, —SO₂H, —OPO₃H₂, —O—CO—(CH₂)ₙ—COOH, —O—(CH₂)ₙ—OCOR, wherein n is 1-4 and R is C₁-C₁₀ alkyl, —NH₂, —NHCOCH₃.

The therapeutic agent (meant in its free state, i.e. before engaging in the present DDS) contains at least one carboxylic group or at least one amino group or at least one hydroxyl group.

When the therapeutic agent contains at least one carboxylic group, the amidic linkage between the agent and 6-NH₂-HA is preferably a direct linkage, with no linker being used.

When the therapeutic agent contains at least one amino or at least one hydroxy group, the drug is preferably attached to 6-NH₂-HA via the linker.

There are no criticality as to the pharmacological class to which the therapeutic active agent belongs. It may be chosen e.g. among analgesic, antihypertensive, anestetic, diuretic, bronchodilator, calcium channel blocker, cholinergic, CNS agent, estrogen, immunomodulator, immunosuppressant, lipotropic, anxiolytic, antiulcerative, antiarrhytmic, antianginal, antibiotic, anti-inflammatory, antiviral, thrombolitic, vasodilator, antipyretic, antidepressant, antipsychotic, antitumour, mucolytic, narcotic antagonist, hormones, anticonvulsant, antihistaminic, antifungal, and antipsoriatic agents, antiproliferative agents, antibiotics. Among them, anti-inflammatory, antibiotic, antitumor drug, more specifically anticancer drugs, are preferred. Example of suitable agents are camptothecin, ibuprofen. methotrexate, taxol, cefazolin, naproxen, lisinopril, penicillinG, nalidixic acid, cholestane, and derivatives thereof.

The linker (meant in its free state, i.e. before engaging in the present DDS) always contains at least one carboxyl group, for linking the 6-NH₂-HA; it also contains at least one other group useful for linking the therapeutic agent, e.g. amino, thiol, further carboxy groups, etc.

Suitable linkers are e.g. linear or branched, aliphatic, aromatic or araliphatic C2-C20 dicarboxylic acids, aminoacids, peptides, linear or branched, aliphatic, aromatic or araliphatic C2-C20 dicarboxylic acid linked to aminoacids, linear or branched, aliphatic, aromatic or araliphatic C2-C20 dicarboxylic acid linked to peptides.

The role of the linker, whenever present, consists in creating an arm or a spacer between the hyaluronic acid and the therapeutic agent. The linker engages, on one side, the 6-NH₂-HA via the amide linkage and, on the other side, the therapeutic agent via any possible covalent-type bond.

When the linker is a dicarboxylic acid linked to aminoacid or to peptide, the carboxylic group forming the amide bond with the HA may be the free acid group of the dicarboxylic acid or that of the aminoacid or that of the peptide.

Preferred linkers are: succinic acid, succinic acid linked to aminoacids, succinic acid linked to peptides.

Preferred aminoacids according to the invention are selected from the group consisting of alanine, valine, leucine, isoleucine, methionine, glycine, serine, cysteine, asparagine, lysine, glutamine, aspartic acid, glutamic acid, proline, histidine, phenylalanine, triptophane and tyrosine. Preferred peptides according to the invention are peptides consisting of different combinations of the above aminoacids or they consists of only one type of aminoacid, they are preferably di-, tri- or tetra-peptides.

As demonstrated in the experimental part, the invented DDSs are characterised by the presence of the therapeutic agent bonded by means of an amidic linkage either directly or by means of a linker to the C6 of the N-acetyl-D-glucosamine units of the hyaluronic acid. No other groups of the HA are involved in the chemical linkage with the drug. This means that both the secondary hydroxyl groups and the carboxyl groups present on hyaluronic acid are free from any drug substitutions and that the DDS of the invention maintain the functionalities present on the polysaccharide as closely as they appear in native HA. So these free functionalities are completely (100%) available for interacting with receptors, such as CD44 or other structures, eg proteoglycans.

The present DDSs are stable and free of undesired reaction by-products and impurities that can be harmful to their practical pharmaceutical use.

The preparation of these DDSs allows to obtain pharmaceutical compounds retaining the pharmacological efficacy of the therapeutic agent. Therefore, they can be successfully used in the treatment of all pathologies responsive to the specific therapeutic agent in the DDS. At the same time they can show some properties that may not have been observed in the therapeutic agent alone, for examples they may show higher affinities for some cells or tissues, they may provide for different bioavailability profiles.

Accordingly, it is a further aspect of the invention the use of the above DDSs in the manufacture of a medicament for the treatment of pathologies appropriate for each therapeutic agent.

It is also an aspect of the invention a pharmaceutical composition containing the DDSs of the invention in admixture with pharmaceutically acceptable excipients and/or diluents. The pharmaceutical composition may be either in the liquid or in solid form; it may be administered through the oral, parenteral, topical, intraarticular route. Systemic administration of the DDS may occurs by intravenous, intraperitoneal, intramuscular, subcutaneous route. Particularly interesting are the injectable pharmaceutical compositions containing the invented DDSs.

A further aspect of the invention is a process for the preparation of the above described DDS. The process includes forming the amide linkage between 6-NH₂-HA and the carboxylic group present on the therapeutic agent (or on the linker); whenever a linker is used, the process also includes the step of linking the drug to the linker, the latter being performed indifferently before or after the amide formation step.

The 6-NH₂-HA can be obtained from HA, by regioselectively introducing an amino group on the C-6 of the N-acetylglucosamine residue of HA. A preferred procedure is exemplified as follows:

a) substituting the hydroxyl group at the C-6 position of the N-acetyl-D-glucosamine units of the hyaluronic acid either in the free form or in the salt form with a leaving group, thus obtaining a 6-activated-HA.
b) converting the 6-activated HA into 6-NH₂-HA.
c) recovering the 6-NH₂-HA.

In step a), the leaving group may be selected from e.g. sulfonate, phosphonate (triphenylphosphonate), cyanide (CN—), nitrite (NO2-), halogen (preferably chloro), sulphate, halogensulfate, nitrate, halogensulfite (chlorosulfite).

The preferred leaving group is halogen, This halogenation can be carried out e.g. as described in WO9918133 and WO0168105, both in the name of the present Applicant. When the leaving group is chloro, the regioselective chlorination is preferably carried out according to the following procedure. The chlorinating reagent such as methanesulphonyl chloride in N,N-dimethylformamide (Vilsmeir Reagent) is added to a solution or suspension of HA in salt form (either sodium form or in an organic base form such as TBA, pyridine or sym-collidine), preferably in the sodium form in N,N-dimethylformamide (DMF) at temperature ranging from −20° C. to −10° C., preferably at −10° C. The reaction temperature is raised from −10° C. to between 40°-65° C., preferably 60° C., over a period of 2 h. The chlorination reaction is then performed at temperature between 40° C. and 65° C., preferably at 60° C., for a period of time comprised between 10 and 24 hours, preferably for 16 h. The reaction is worked up by treatment with saturated aqueous NaHCO₃ solution to achieve pH 8 and then by treatment with aqueous NaOH to pH 9; this step allows to remove the formate ester groups formed during the reaction at the secondary hydroxyl groups of the HA molecule. The reaction mixture is then neutralised by addition of diluted HCl. The desired 6-chloro-hyaluronic acid is then recovered by means of standard techniques.

Other preferred leaving groups are sulfonates such as an alkyl- or aryl-sulfonates, resulting in 6-sulfonated-HA; among sulphonates, methansulphonate is preferred. The regioselective sulfonylation is carried out using as sulfonylating reagent an alkyl- or aryl-sulfonyl halide, preferably chloride, in presence of an organic or inorganic base, preferably an organic base. The alkyl- or aryl-sulfonyl halide may be chosen among, preferred are methylsulfonyl (mesyl), toluene-p-sulfonyl (tosyl), trifyl, trimsyl, tripsyl, 1,1-sulfonyl-imidazole. The organic base is selected preferably among the different organic amines, such as diisopropylethylamine, triethylamine.

The solvent is chosen from the group consisting of: dimethylformamide, dimethylacetamide, dimethylsulfoxide, formamide. The general sulfonylation procedure is as follows. The base, preferably organic base is added to a suspension or a solution of HA in salt form, preferably in an organic base form, by stirring under nitrogen flux. Then the alkyl- or aryl-sulfonyl chloride in a suitable solvent, preferably the same solvent, is added dropwise. After a period of time ranging from 2 to 90 minutes (preferably 45-75 min), the reaction is quenched by addition of NaHCO₃ to remove the formate ester groups formed during the reaction at secondary hydroxyl groups of HA. Then the reaction is allowed to continue for about 10-20 hours, preferably 18 hours. The reaction product (6-sulfonated-HA) is either directly recovered form the solution by means of known techniques, such as precipitation, drying or before recovery the solution is treated in such a way as to allow the obtainment of the 6-sulfonated-HA in a suitable salt form, such as HA-6-sulfonated:TBA.

According to step b), the leaving group at the C6 position is displaced to afford the intermediate 6-NH₂-HA compound. Step b) may be carried out by treating the 6-activated-HA with concentrated ammonia at high temperature (e.g. 40-70° C., preferably 60-80° C.) for 2-50 hours thereby obtaining a 6-NH₂-HA. In particular, when the leaving group is chloro then the 6-amino-HA is prepared by treatment of 6-Cl-HA either as an inorganic salt or an organic salt, preferably sodium or tetrabutylammonium (TBA) salt, respectively, with or without DMSO, with concentrated (25%) ammonia at 80° C. for periods of 7 to 48 hours, depending upon the degree of substitution required. Alternatively, when the leaving group is mesylate the intermediate 6-amino-HA is prepared by treatment of 6-mesylate-HA either as an inorganic salt or an organic salt, preferably sodium or tetrabutylammonium (TBA) salt, respectively, with concentrated (25%) ammonia at 60° C. for 18 hours to give complete conversion of mesylate groups to amino groups.

The synthesis of the 6-NH₂-HA intermediate could also be obtained stepwise, by substitution of chloride in 6-Cl-HA or mesylate in 6-OMs-HA by azide anion, followed by reduction. 6-Cl-HA requires stronger conditions, so substitution is carried out in dimethylsufoxide and a crown ether is used to enhance azide nucleophilicity. 6-OMs-HA shows a higher reactivity giving complete conversion using water as a solvent and sodium azide as the nucleophile. The reduction stage is also performed in water, avoiding any need for salt exchange. Several methods can be used for reducing azides in aqueous conditions such as dithiotreitol in physiological buffers, hydrogen sulfide in aqueous pyridine, zinc and ammonium chloride in water/alcohol mixtures, copper salts and sodium borohydride in water, catalytic hydrogenation in water. Sulfur-containing compounds are regarded as a second choice, and the preferred method entails the use of divalent copper salts and sodium borohydride in water (Fringuelli J. Org. Chem. 2003, 68, 7041-7045) and the classical catalytic hydrogenation, which is carried out in water using ammonium formate as a hydrogen donor and Pd/C as a catalyst. The reduction with zinc and ammonium chloride can also be used since it also provides for positive Kaiser test but problems in the purification stage may occurs.

Step c) is carried out according to techniques well known to the expert of the field. The part of the process which includes steps a) to c) is regioselective i.e. it occurs by substituting the sole primary hydroxyl groups which are on the C6 position of the HA; no other hydroxyl groups are substituted.

The amide-forming step between 6-NH₂-HA and the therapeutic agent (or linker), is performed under standard reaction conditions for this reaction, as exemplified in the experimental part. When the linker is used, the therapeutic agent may be bonded to the linker before or after the amide formation step with 6-NH₂-HA, preferably before.

When the linker is bonded to the therapeutic agent before the amide-formation step, the latter ends directly with the final DDS of the invention, having structure HA-6-NHCO-linker-therapeutic agent.

When the linker is bonded to the therapeutic after the amide-formation step, the latter ends with the intermediate compound of structure HA-6-NHCO-linker, which is then reacted with the therapeutic agent obtaining the final DDS having structure HA-6-NHCO-linker-therapeutic agent.

When the linker is not used, the amide-formation step ends with the final DDS having structure HA-6-NHCO-therapeutic agent.

The type of bond between linker and therapeutic agent is not critical and can be obtained by any suitable chemical reaction forming a covalent bond; esters, ethers, secondary/tertiary amines, are examples of such bonding.

In particular, when the therapeutic agent contains a hydroxy group and the linker contains a carboxylic group (additional to that involved in the amide linkage), the agent-linker bond is conveniently of ester type: thus the therapeutic agent containing the hydroxyl function (e.g. camptothecin, taxol) is treated with the linker (e.g. succinic acid) in a chlorinated organic solvent (.e. methylene chloride), obtaining an agent-linker monoester (e.g. hemisuccinate); the resulting monoester is activated (e.g. using N-hydroxysuccinimide (NHS) using diisopropylcarbodiimide (DIPC) in DMSO at ambient temperature) and reacted with 6-NH₂-HA, to give the DDS of the invention.

In another embodiment, when the therapeutic agent contains a hydroxy group and the linker includes an aminoacid or a peptide, the agent may be linked: (i) to the amine or (ii) to the carboxylic function of said aminoacid/peptide; in this way it is possible to achieve the cellular drug release conditions either assisted by enzymatic or pH assisted hydrolysis.

In the case (i), the linkage may be obtained by treatment of the therapeutic agent containing the hydroxylic function (such as camptothecin, CPT) with the N-protected aminoacid/peptide (such as Cbz-glycine-OH), to give the corresponding ester derivative followed by the regeneration of the amino group. Then the resulting product having the free NH₂ group is treated with C2-C20 dicarboxylic acid to give the corresponding monoester. The agent-linker monoester is then reacted with 6-NH₂-HA affording the final DDS.

In the case (ii), the reaction sequence involves: reacting the therapeutic agent containing the hydroxylic function (e.g. camptothecin, CPT) with C2-C20 dicarboxylic acid (e.g. succinic acid) to give the corresponding monoester (e.g. hemisuccinate); the monoester is then treated with an aminoacid (e.g. glycine) or a peptide, in which the carboxyl group is protected; after removal of the protecting group and treatment with 6-NH₂-HA, the final DDS is obtained.

When the therapeutic agent contains a carboxyl group, this can be directly linked to the N atom on C6 position of the 6-NH₂-HA via amidic linkage, without using any linkers, obtaining the final DDS with structure HA-6-NHCO-therapeutic agent. Examples of therapeutic agents having a carboxyl function are methotrexate, an antitumour drug, or ibuprofen, an anti-inflammatory drug. These agents may also be attached to 6-NH₂-HA via linker. In this case, an amidic linkage is first formed between the N atom on C6 position of the 6-NH₂-HA and the carboxylic function of the linker; the compound HA-6-NHCO-linker thus obtained is then reacted with the therapeutic agent, obtaining the final DDS.

The reaction conditions on the invention are mild and allow to obtain a final DDS which is stable and free of undesired by-products and impurities that can be harmful to its practical pharmaceutical use.

The invention is now illustrated by the following non limiting examples.

### EXPERIMENTAL PART

### Abbreviations Used

HA: hyaluronic acid. TBA: tetrabutylammonium. DMF: dimethylformamide. DMSO: dimethylsulfoxide. DIEA: N,N-diisopropylethylamine. DMAP: 4-dimethylaminopyridine. DCM: dichloromethane. DIPC: diisopropylcarbodiimide. TFA: trifluoroacetic acid. THF: tetrahydrofuran. MeOH: methanol. EtOH: ethanol. CPT: camptothecin. MTX: methotrexate. TXL: taxol. EDC: N-(3-Dimethylaminopropyl)-N′-ethylcarbodiimide. Cbz: benzyloxycarbonyl. Boc: tert-butoxycarbonyl. HOBt: 1-hydroxybenzotriazole. NHS: N-hydroxysuccinimide. EtOAc: ethyl acetate.

The structures of 6-Cl-HA, 6-OMs-HA, 6-NH₂-HA and all the described derivatives of general formula HA-6-NH-acyl, HA-6-O-acyl and HA-O-acyl were supported by NMR. ¹H NMR, ¹H DOSY, ¹³C NMR, HSQC spectra confirmed the covalent linkage of the drugs on C6 position of 6-deoxy-6-amino-N-acetyl-D-glucosamine for all DDS of the type HA-6-NH-acyl.

NMR spectra were taken on a Varian Inova 500 spectrometer, equipped with a linear gradient along the z axis and on a Varian Mercury 200 spectrometer, in D₂O for HA derivatives and as specified for other intermediates.

Hyaluronic acid of MW 20.000 was used as starting material, unless otherwise noted.

The TBA salt of hyaluronic acid was prepared by ion exchange. Briefly, Amberlite IRA-120 resin was treated with excess 20% tetrabutylammonium hydroxide solution for 24 h, then it was washed with water. A solution of HA in water (5%) was then gently mixed with the resin for 24 h. Filtration, concentration and freeze-drying afforded HA TBA salt with stoichiometric TBA content, as confirmed by proton NMR.

### Example 1

The determination of chloride content in 6-Cl-HA by NMR was achieved by integration of the ¹³C NMR peak at 60.5 ppm (CH₂OH) versus the peak at 44.0 ppm (CH₂Cl), using a quantitative pulse sequence.

### Example 2

The determination of mesylate content in the HA-6-Mesylate (6-Ms-HA) by NMR was achieved by integration of the peaks in the region 3.10÷3.32 ppm (1H of HA chain and 3H of mesylate) versus the peak at 1.95 ppm (3H of HA chain). Selectivity for the C6 position was confirmed by ¹³C NMR and HSQC NMR spectra: secondary mesylates were not detected.

### Example 3

The determination of amine content in 6-NH₂-HA by NMR was achieved by integration of the ¹³C NMR peaks at 60.5 ppm (CH₂OH), at 44.0 ppm (left CH₂Cl, present only when 6-NH₂-HA is made from 6-Cl-HA) and at 40.5 ppm (CH₂NH₂), using a quantitative pulse sequence.

### Example 4

The determination of azide content in 6-N₃-HA compounds was determined by ¹³C NMR, integrating the signal of CH₂—OH at 60.5 ppm versus the signal of CH₂—N₃ at 51 ppm.

### Example 5

The determination of the CPT content in DDS was achieved combining a termogravimetric analysis for the determination of the water content with an HPLC method for the determination of free and bound CPT. Analytical parameters for termogravimetric water content determination in DDS are.

| | |
|---|---|
| Thermogravimetric balance: | TGA Perkin Elmer |
| Atmosphere: | Nitrogen |
| Gas flow | Furnace flow: 25 mL/min; Balance flow: 50 |
| | mL/min |
| Temperature range | 50-250° C. |
| Temperature scan rate | 10° C./min |

To determine free camptothecin, a 1:1 water/methanol solution of conjugate was injected. Total camptothecin was determined after hydrolysis. Hydrolysis was carried out for 2 hours in sodium hydroxide 0.1 M at room temperature, under stirring. The samples were subsequently added of a methanol amount to obtain a final methanol concentration (concentration prior to the injection), of 50%, to prevent camptothecin precipitation, then neutralized with 1M HCl solution and finally added of KH₂PO₄ 25 mM buffer, pH 2.5, up to the final volume. Bound camptothecin was obtained subtracting free camptothecin from total camptothecin.

Analytical parameters for HPLC determination of CPT in DDS are:

| | |
|---|---|
| Cromatographic | Agilent 1100 series |
| system | |
| Column set: | Column name: Merck Chromolith RP-18e |
| | Column size: 100 × 4.60 mm |
| | Temperature: 40° C. |
| Guard column: | Guard column description: Merck Guard Cartridge |
| | RP-18e |
| | Guard column size: 5 × 4.6 mm; Temperature: 40° C. |
| Eluent A: | Methanol |
| Eluent B: | KH₂PO₄ 25 mM buffer pH 2.5 |
| Mobile phase | 0′ 35% A + 65% B; |
| gradient | 15′ 55% A + 45% B; |
| | 20′ 55% A + 45% B |
| Flow rate: | 1 mL/min |
| Detectors: | UV-VIS (λ = 370 nm); Fluorimeter (Excitation λ = |
| | 380 nm, Emission λ = 440 nm) |
| Run time: | 20 min |
| Injection volume: | 10 μL |

### Example 6

### Determination of Weight Average Molecular Weight (Mw)

The molecular weight of the hyaluronic acid DDS was measured by HP-SEC (High Performance Size Exclusion Chromatography). The analysis conditions were: Chromatograph: HPLC pump 980-PU (Jasco Ser. No. B3901325) with Rheodyne 9125 injector. Column: TSK PWxI (TosoBioscience) G6000+G5000+G3000 6, 10, 13 μm particle size; Temperature: 40° C. Mobile phase: NaCl 0.15 M+0.01% NaN₃. Flux: 0.8 mL/min. Detector: MALLS (WYATT DAWN EOS—WYATT, USA), λ=690 nm, (dn/dc=0.167 mL/g), UV spectrophotometric detector 875-UV (Jasco, Ser. No. D3693916), λ=305 nm, Interferometric Refractive Index OPTILAB REX (WYATT, USA); λ=690 nm, Sensitivity: 128×; Temperature: 35° C. Injected volume:100 μl, run time 60 minutes.

The samples to be analysed were solubilised in 0.9% NaCl at the concentration of about 1.0 mg/ml and kept under stirring for 12 hours. Then, the solutions were filtered on a 0.45 μm porosity filter (Sartorius Minisart RC25 17795Q) and finally injected in the chromatograph. The analysis allows the measurement of Mw (weight average molecular weight), Mn (number average molecular weight), PI (polydispersity). The concentration of the polymeric samples solutions were controlled by means of the integral of the refractive index.

### Example 7

### Preparation of 6-Cl-HA Sodium Salt

50 g of hyaluronan sodium salt were suspended in 900 mL of dry dimethylformamide under nitrogen, with mechanical stirring at 20° C. The suspension was then cooled to −10° C. and 97 mL of methanesulfonyl chloride were added during 30 min. After additional 30 min at −10° C., the temperature was raised to 20° C. After 1 h the temperature was gradually raised (during 1 h) to 60° C. and stirring was continued for 18.5 h. The reaction mixture was then poured in portions into a mixture of ice and sodium carbonate solution (4 L, initial pH=11) with vigorous mixing, maintaining the pH around 9 by addition of 1.5 M NaOH when required. The resulting brownish suspension (final volume 6 L) was stirred at pH 9.5 at room temperature for about 48 h, whereupon a clear solution formed. This was filtered to remove solids and then ultrafiltered (10 KDa cut-off membrane). The resulting solution was concentrated in a rotary evaporator to a final volume of about 1 litre and freeze-dried to afford 34.9 g of 6-Cl-HA sodium salt as an off-white solid (DS 66% mol/mol, determined by ¹³C NMR). ¹³C NMR ppm: 22.6, 44.0, 54.4, 60.7, 68.6, 69.3, 72.6, 73.8, 74.1, 75.5, 76.3, 80.1, 80.9, 82.3, 101.0, 103.4, 174.0, 174.1, 175.0.

### Example 8

### Preparation of 6-Cl-HA Sodium Salt

Following the same procedure of Example 7, starting from 50 g of hyaluronan sodium salt and maintaining the heating at 60° C. for 12 h, 33.5 g of 6-Cl-HA sodium salt were obtained as an off-white solid (DS 38% mol/mol, determined by ¹³C NMR).

### Example 9

### Preparation of 6-Cl-HA Sodium Salt

Following the same procedure of Example 7, starting from 50 g of hyaluronan sodium salt and maintaining the heating at 60° C. for 7 h, 33.1 g of 6-Cl-HA sodium salt were obtained as an off-white solid (DS 16% mol/mol, determined by ¹³C NMR).

### Example 10

### Preparation of 6-Cl-HA Sodium Salt

Following the same procedure of Example 7, starting from 50 g of hyaluronan sodium salt and maintaining the heating at 60° C. for 5 h, 32.4 g of 6-Cl-HA sodium salt were obtained as an off-white solid (DS 8% mol/mol, determined by ¹³C NMR).

### Example 11

### Preparation of 6-O-Methanesulfonylhyaluronic Acid Sodium Salt (6-OMs-HA)

To a solution of 20.03 g (32.3 mmol) of TBA salt of HA in 500 ml of DMF were added 4.86 ml (28.4 mmol) of DIEA by stirring under nitrogen at −10° C. MsCl (1001 μL; 12.9 mmol) was then added dropwise and the resulting mixture was stirred for 1 h at −10° C. The reaction mixture was quenched by adding saturated NaHCO₃ solution (1 L) and bringing the total volume to 3 L with water (resulting pH: 9); stirring was maintained overnight. The resulting solution was ultrafiltered and concentrated in a rotary evaporator. The solution was freeze-dried to afford 11.12 g of a white solid. Total mesylate DS 12.3% mol/mol by proton NMR.

### Example 12

### Preparation of 6-O-Methanesulfonylhyaluronic Acid Sodium Salt (6-OMs-HA)

To a solution of 1.00 g (1.61 mmol) of TBA salt of HA in 40 ml of DMF were added 381 μL (2.24 mmol) of DIEA by stirring under nitrogen at −10° C. MsCl (79 μL; 1.01 mmol) was then added dropwise and the resulting mixture was stirred for 1 h at −10° C. The reaction mixture was quenched by adding saturated NaHCO₃ solution (50 ml) and bringing the total volume to 200 ml with water (resulting pH: 9); stirring was maintained overnight. The resulting solution was ultrafiltered and concentrated in a rotary evaporator. The solution was freeze-dried to afford 588 mg of a white solid. Total mesylate DS 15% mol/mol by proton NMR.

### Example 13

### Preparation of 6-O-Methanesulfonylhyaluronic Acid Sodium Salt (6-OMs-HA)

To a solution of 42.2 g (68.1 mmol) of TBA salt of HA in 1.00 L of DMF were added 25.6 ml (149.8 mmol) of DIEA by stirring under nitrogen at −10° C. MsCl (5.29 ml; 68.1 mmol) was then added dropwise and the resulting mixture was stirred for 1 h at −10° C. The reaction mixture was quenched by adding saturated NaHCO₃ solution (2 L) and bringing the total volume to 5.5 L with water (resulting pH: 9); stirring was maintained overnight. The resulting solution was ultrafiltered and concentrated in a rotary evaporator. The solution was freeze-dried to afford 26.0 g of a white solid. Total mesylate DS 18% mol/mol by proton NMR.

### Example 14

### Preparation of 6-O-Methanesulfonylhyaluronic Acid Sodium Salt (6-OMs-HA)

To a solution of 20.0 g (32.3 mmol) of TBA salt of HA in 500 ml of DMF were added 9.12 ml (53.3 mmol) of DIEA by stirring under nitrogen at −10° C. MsCl (3.76 ml; 48.4 mmol) was then added dropwise and the resulting mixture was stirred for 1 h at −10° C. The reaction mixture was quenched by adding saturated NaHCO₃ solution (1 L) and bringing the total volume to 3 L with water (resulting pH: 9); stirring was maintained overnight. The resulting solution was ultrafiltered and concentrated in a rotary evaporator. The solution was freeze-dried to afford 11.12 g of a white solid. Total mesylate DS 30% mol/mol by proton NMR.

### Example 15

### Preparation of 6-O-Methanesulfonylhyaluronic Acid Sodium Salt (6-OMs-HA)

To a solution of 20.0 g (32.3 mmol) of TBA salt of HA in 500 ml of DMF were added 9.12 ml (53.3 mmol) of DIEA by stirring under nitrogen at −10° C. MsCl (3.76 ml; 48.4 mmol) was then added dropwise and the resulting mixture was stirred for 1 h at −10° C. The reaction mixture was quenched by adding saturated NaHCO₃ solution (1 L) and bringing the total volume to 3 L with water (resulting pH: 9); stirring was maintained overnight. The resulting solution was ultrafiltered and concentrated in a rotary evaporator. The solution was freeze-dried to afford 11.12 g of a white solid. Total mesylate DS 30% mol/mol by proton NMR.

### Example 16

### Preparation of 6-NH₂-HA TBA Salt

5.0 g of 6-Cl-HA from Example 7 were dissolved in 100 ml of conc. NH₄OH solution, in a reactor suitable for reactions under pressure. The reactor was sealed and the solution was heated at 80° C. for 21 h, then it was cooled and excess ammonia was removed under vacuum. After ultrafiltration, the solution was treated with amberlite IRA-120 loaded with TBA. Then it was freeze-dried to afford 5.43 g of 6-NH₂-HA TBA salt as an off-white solid (DS 33% mol/mol, determined by ¹³C NMR). MW: 17.220, P.I. 1.8. ¹³C NMR ppm (TBA signals not included): 22.6, 40.5, 44.0, 54.4, 60.7, 68.6, 69.3, 70.5, 72.0, 72.6, 73.8, 75.5, 76.3, 78.2, 80.1, 80.9, 82.3, 99.7, 101.0, 103.4, 174.0, 174.1, 175.0.

### Example 17

### Preparation of 6-NH₂-HA TBA Salt

5.0 g of 6-Cl-HA from Example 7 were dissolved in 100 ml of conc. NH₄OH solution, in a reactor suitable for reactions under pressure. The reactor was sealed and the solution was heated at 80° C. for 40 h, then it was cooled and excess ammonia was removed under vacuum. After ultrafiltration, the solution was treated with amberlite IRA-120 loaded with TBA. Then it was freeze-dried to afford 4.34 g of 6-NH₂-HA TBA salt as an off-white solid (DS 42% mol/mol, determined by ¹³C NMR).

### Example 18

### Preparation of 6-NH₂-HA TBA Salt

5.0 g of 6-Cl-HA from Example 7 were dissolved in 100 ml of conc. NH₄OH solution, in a reactor suitable for reactions under pressure. The reactor was sealed and the solution was heated at 80° C. for 48 h, then it was cooled and excess ammonia was removed under vacuum. After ultrafiltration, the solution was treated with amberlite IRA-120 loaded with TBA. Then it was freeze-dried to afford 4.05 g of 6-NH₂-HA TBA salt as an off-white solid (DS 50% mol/mol, determined by ¹³C NMR).

### Example 19

### Preparation of 6-NH₂-HA TBA Salt

5.0 g of 6-Cl-HA from Example 8 were dissolved in 100 ml of conc. NH₄OH solution, in a reactor suitable for reactions under pressure. The reactor was sealed and the solution was heated at 80° C. for 22 h, then it was cooled and excess ammonia was removed under vacuum. After ultrafiltration, the solution was treated with amberlite IRA-120 loaded with TBA. Then it was freeze-dried to afford 6.51 g of 6-NH₂-HA TBA salt as an off-white solid (DS 20% mol/mol, determined by ¹³C NMR). MW: 15.410, P.I. 1.5.

### Example 20

### Preparation of 6-NH₂-HA TBA Salt

5.0 g of 6-Cl-HA from Example 8 were dissolved in 100 ml of conc. NH₄OH solution, in a reactor suitable for reactions under pressure. The reactor was sealed and the solution was heated at 80° C. for 38 h, then it was cooled and excess ammonia was removed under vacuum. After ultrafiltration, the solution was treated with amberlite IRA-120 loaded with TBA. Then it was freeze-dried to afford 5.90 g of 6-NH₂-HA TBA salt as an off-white solid (DS 25% mol/mol, determined by ¹³C NMR). MW: 16.370, P.I. 1.6.

### Example 21

### Preparation of 6-NH₂-HA TBA Salt

5.0 g of 6-Cl-HA from Example 9 were dissolved in 100 ml of conc. NH₄OH solution, in a reactor suitable for reactions under pressure. The reactor was sealed and the solution was heated at 80° C. for 22 h, then it was cooled and excess ammonia was removed under vacuum. After ultrafiltration, the solution was treated with amberlite IRA-120 loaded with TBA. Then it was freeze-dried to afford 6.43 g of 6-NH₂-HA TBA salt as an off-white solid (DS 13% mol/mol, determined by ¹³C NMR). MW: 14.420, P.I. 1.4.

### Example 22

### Preparation of 6-NH₂-HA TBA Salt

5.0 g of 6-Cl-HA from Example 9 were dissolved in 100 ml of conc. NH₄OH solution, in a reactor suitable for reactions under pressure. The reactor was sealed and the solution was heated at 80° C. for 7 h, then it was cooled and excess ammonia was removed under vacuum. After ultrafiltration, the solution was treated with amberlite IRA-120 loaded with TBA. Then it was freeze-dried to afford 5.95 g of 6-NH₂-HA TBA salt as an off-white solid (DS 4% mol/mol, determined by ¹³C NMR). MW: 13.960, P.I. 1.4.

### Example 23

### Preparation of 6-NH₂-HA TBA Salt

5.0 g of 6-Cl-HA from Example 10 were dissolved in 100 ml of conc. NH₄OH solution, in a reactor suitable for reactions under pressure. The reactor was sealed and the solution was heated at 80° C. for 7 h, then it was cooled and excess ammonia was removed under vacuum. After ultrafiltration, the solution was treated with amberlite IRA-120 loaded with TBA. Then it was freeze-dried to afford 6.22 g of 6-NH₂-HA TBA salt as an off-white solid (DS 2% mol/mol, determined by ¹³C NMR). MW: 13.680, P.I. 1.4.

### Example 24

### Preparation of 6-NH₂-HA TBA Salt

100 mg of 6-OMs-HA from Example 15 were dissolved in 3 ml of conc. NH₄OH solution, in a sealable flask. The solution was heated at 60° C. for 18 h, then it was cooled and excess ammonia was removed under vacuum. After ultrafiltration, the solution was treated with amberlite IRA-120 loaded with TBA. Then it was freeze-dried to afford 132 mg 6-NH₂-HA TBA salt as a white solid (DS 30% mol/mol, determined by ¹³C NMR).

¹³C NMR ppm (TBA signals not included): 22.3, 40.5, 53.9, 60.4, 68.1, 69.8, 71.0, 71.8, 72.4, 74.7, 77.2, 79.5, 82.0, 99.5, 100.8, 103.0, 173.0, 173.9, 174.3.

### Example 25

### Preparation of 6-NH₂-HA TBA Salt

10 g of 6-OMs-HA from Example 14 were dissolved in 200 ml of conc. NH₄OH solution, in a reactor suitable for reactions under pressure. The reactor was sealed and heated at 60° C. for 18 h, then it was cooled and excess ammonia was removed under vacuum. After neutralization with HCl solution and ultrafiltration, the solution was treated with amberlite IRA-120 loaded with TBA. Then it was freeze-dried to afford 12.8 g of 6-NH₂-HA TBA salt as a white solid (DS 20% mol/mol, determined by ¹³C NMR).

### Example 26

### Preparation of 6-NH₂-HA TBA Salt

10 g of 6-OMs-HA from Example 13 were dissolved in 200 ml of conc. NH₄OH solution, in a reactor suitable for reactions under pressure. The reactor was sealed and heated at 60° C. for 18 h, then it was cooled and excess ammonia was removed under vacuum. After neutralization with HCl solution and ultrafiltration, the solution was treated with amberlite IRA-120 loaded with TBA. Then it was freeze-dried to afford 12.6 g of 6-NH₂-HA TBA salt as a white solid (DS 12% mol/mol, determined by ¹³C NMR).

### Example 27

### Preparation of 6-NH₂-HA TBA Salt

500 g of 6-OMs-HA from Example 12 were dissolved in 15 ml of conc. NH₄OH solution, in a sealable flask. The solution was sealed and heated at 60° C. for 18 h, then it was cooled and excess ammonia was removed under vacuum. After neutralization with HCl solution and ultrafiltration, the solution was treated with amberlite IRA-120 loaded with TBA. Then it was freeze-dried to afford 628 mg of 6-NH₂-HA TBA salt as a white solid (DS 8% mol/mol, determined by ¹³C NMR).

### Example 28

### Preparation of 6-NH₂-HA TBA Salt

10 g of 6-OMs-HA from Example 11 were dissolved in 200 ml of conc. NH₄OH solution, in a reactor suitable for reactions under pressure. The reactor was sealed and heated at 60° C. for 18 h, then it was cooled and excess ammonia was removed under vacuum. After neutralization with HCl solution and ultrafiltration, the solution was treated with amberlite IRA-120 loaded with TBA. Then it was freeze-dried to afford 12.9 g of 6-NH₂-HA TBA salt as a white solid (DS 6% mol/mol, determined by ¹³C NMR).

### Example 29

### Preparation of 6-NH₂-HA Sodium Salt

100 mg of 6-OMs-HA from Example 15 were dissolved in 3 ml of conc. NH₄OH solution, in a sealable flask. The solution was heated at 60° C. for 18 h, then it was cooled and excess ammonia was removed under vacuum. The solution was treated with 0.5 ml of saturated sodium chloride and stirred for 30 min. Then it was dialysed and freeze-dried to afford 92 mg of 6-NH₂-HA sodium salt as a white solid (DS 30% mol/mol, determined by ¹³C NMR).

### Example 30

### Preparation of 6-N₃-HA Sodium Salt

1.00 g (2.38 mmol) of 6-Cl-HA sodium salt from Example 8 were converted to the correspondent TBA salt by dissolving in water and treating with amberlite IRA-120 loaded with TBA. The resulting solution was freeze-dried to afford 1.53 g of 6-Cl-HA TBA salt. This solid was dissolved in 30 ml of DMSO and 5.0 g of sodium azide and 1.0 g of 18-crown-6 were added, heating to 80° C. and then stirring for 24 h. The mixture was poured into water, the resulting solution was ultrafiltered and freeze-dried to give 820 mg of a solid. The DS in azide was 30% by ¹³C NMR.

### Example 31

### Preparation of 6-N₃-HA Sodium Salt

200 mg (0.5 mmol) of 6-OMs-HA sodium salt from Example 14 were dissolved in 4 ml of water. 1.20 g of sodium azide were added and the solution was stirred at 80° C. for 16 h. Dialysis against water and freeze-drying afforded 180 mg of a white solid. The DS in azide was 15% by ¹³C NMR. No residual mesylate was observed in proton NMR.

### Example 32

### Preparation of 6-N₃-HA Sodium Salt

2.0 g (4.8 mmol) of 6-OMs-HA sodium salt from Example 20 were dissolved in 40 ml of water. 10.0 g of sodium azide were added and the solution was stirred at 80° C. for 16 h. Dialysis against water and freeze-drying afforded 1.65 g of a white solid. The DS in azide was 10% by ¹³C NMR. No residual mesylate was observed in proton NMR.

### Example 33

### Preparation of 6-NH₂-HA Sodium Salt

To a solution of 150 mg (0.37 mmol) of 6-N₃-HA sodium salt from Example 32 and 16 mg (0.1 mmol) of CuSO₄ in 3 ml of water, were added portionwise, stirring at 0° C., 38 mg (1.0 mmol) of NaBH₄. Initial gas evolution was observed and a black mixture formed. After 1 h the mixture was treated with conc. NH₄OH to pH=10 and filtered through celite. The resulting solution was acidified to pH=9 with 1N HCl, whereupon a black precipitate formed which was filtered off using a short celite pad. The resulting solution was dialysed against water and freeze-dried to afford 135 mg of a white solid. The DS of amino groups was 10% by ¹³C NMR and no residual azide was observed.

### Example 33

### Preparation of 6-NH₂-HA Sodium Salt

A solution of 200 mg (0.50 mmol) of 6-N₃-HA sodium salt from Example 32 and 120 mg of ammonium formate in 4 ml of water was purged from air by several nitrogen/vacuum cycles. Then, under nitrogen, 40 mg of 5% Pd/C were added and the mixture was stirred at room temperature for 18 h. Then it was filtered through a short pad of celite, dialysed against water and freeze-dried to afford 184 mg of a white solid. The DS of amino groups was 10% by ¹³C NMR and no residual azide was observed.

### Example 34

### CPT-20-O-hemisuccinate

To a solution of 2.98 g (17.1 mmol) of succinic acid mono-tert-butyl ester and 1.40 g (11.5 mmol) of p-dimethylamino-pyridine in 200 ml of dichloromethane were added, while stirring at room temperature, 2.68 ml (17.3 mmol) of diisopropylcarbodiimide and 3.00 g (8.62 mmol) of CPT. After stirring overnight, the resulting suspension was diluted with 80 ml of dichloromethane to obtain a solution which was washed with 0.1N HCl solution and dried over anhydrous sodium sulfate. Then it was filtered and evaporated to dryness in a rotary evaporator. The residue was crystallized with 100 ml of MeOH, filtered and washed with MeOH. After drying on the filter, the solid was treated with 50 ml of a 40% v/v solution of trifluoroacetic acid in dichloromethane and, after 1 h standing at room temperature, the resulting greenish solution was evaporated to dryness in a rotary evaporator. The residue was crystallized with 100 ml of MeOH, filtered and washed with MeOH and diethyl ether. After drying in vacuo, 3.67 g (8.19 mmol, 95%) of a pale yellow solid were obtained.

### Example 35

### HA-6-NHCO(CH₂)₂—CO-20-O-CPT Sodium Salt

To a solution of 56 mg (0.124 mmol) of CPT-20-O-hemisuccinate from Example 34 and 17.3 mg (0.150 mmol) of N-hydroxysuccinimide in 3 ml of dimethylsulfoxide were added, with stirring under nitrogen at room temperature, 19 μL (0.124 mmol) of diisopropylcarbodiimide. After 16 h, 77 mg (0.124 mmol) of 6-NH₂-HA TBA salt from example 20 were added, and stirring was continued for 5 h. 0.15 ml of saturated NaCl solution were then added and stirring was continued for 30 min. The mixture was poured into 10 ml of EtOH while stirring, the resulting slurry was stirred for 10 min and then filtered and washed with EtOH. The solid was suspended in 10 ml of dimethylformamide, slurried for 30 min, filtered and washed once with dimethylformamide and twice with MeOH. After drying on the filter, the solid was dissolved in 10 ml of water and dialysed against water. Then the solution was filtered through a 0.22μ pore size membrane and freeze-dried to give 60 mg of a white solid. DS in CPT by proton NMR: 25% mol/mol.

### Example 36

### Reaction Between HA TBA Salt and Activated CPT-20-O-hemisuccinate

To a solution of 56 mg (0.124 mmol) of CPT-20-O-hemisuccinate from Example 34 and 17.3 mg (0.150 mmol) of N-hydroxysuccinimide in 3 ml of dimethylsulfoxide were added, with stirring under nitrogen at room temperature, 19 μL (0.124 mmol) of diisopropylcarbodiimide. After 16 h, 77 mg (0.124 mmol) of HA TBA salt were added, and stirring was continued for 5 h. 0.15 ml of saturated NaCl solution were then added and stirring was continued for 30 min. The mixture was poured into 10 ml of EtOH while stirring, the resulting slurry was stirred for 10 min and then filtered and washed with EtOH. The solid was suspended in 10 ml of dimethylformamide, slurried for 30 min, filtered and washed once with dimethylformamide and twice with MeOH. After drying on the filter, the solid was dissolved in 10 ml of water and dialysed against water. Then the solution was filtered through a 0.22μ pore size membrane and freeze-dried to give 48 mg of a white solid.

DS in CPT by proton NMR: 0% mol/mol.

### Example 37

### Structure Confirmation of HA-6-NHCO(CH₂)₂—CO-20-O-CPT from Example 35 by Means of NMR

The proton spectrum in D₂O of HA-6-NHCO(CH₂)₂—CO-20-O-CPT from Example 35 shows a pattern of very broad signals that can be attributed to bound CPT-hemisuccinate. A DOSY weighed spectrum confirmed that the new signals belong to a species bonded to hyaluronan chain. The two doublets present between 5.5 and 5.8 ppm can be attributed to the lactone of CPT and integrate correctly with respect to other signals belonging to bonded CPT. An HSQC spectrum confirmed this attribution and allowed the complete identification of all CPT-hemisuccinate signals and of the newly formed amidic 6-CH₂ on the polymer.

Quantification of bonded CPT was estimated to be 25% mol/mol by ¹H NMR. The DS was confirmed by adding 2 mg of solid LiOH monohydrate to the NMR tube. After a few minutes a new proton spectrum was taken, which showed complete hydrolysis of CPT from the conjugate; the signals of the lithium salt of the open ring form of CPT could be integrated against HA, confirming the DS to be 25% mol/mol. In this spectrum the signals of the succinate chain are seen as two broad multiplets at two different chemical shifts for the two methylene groups, indicating that succinate is still bonded to the polymer; this was confirmed by a DOSY weighed spectrum, and after a week at pH 13, NMR still showed that hemisuccinate was bonded on the polymer.

In starting 6-NH₂-HA from Example 20 the amine DS was estimated to be 25% mol/mol by ¹³C NMR, and, from the HSQC spectrum of HA-6-NHCO(CH₂)₂—CO-20-O-CPT, no traces of 6-CH₂NH₂ are left on the polymer.

These experiments show that CPT-20-O-hemisuccinate binds exclusively through amide bonds to the amine of 6-NH₂-HA in the conjugation step; this was confirmed by an independent reaction (Example 36) where native HA TBA salt was used and no bonded CPT was found.

Moreover, bonded CPT is present as its lactone form (from proton and carbon chemical shifts and from proton integrations); for comparison, spectra of CPT were taken in basic water, where CPT is soluble as the open lactone carboxylate. In this case the proton and carbon chemical shifts of the open lactone are unambiguously different.

Finally, 1D and 2D spectra of pure open ring CPT (taken in basic H₂O and using water suppressing techniques) can be superimposed on the spectra of the hydrolysed conjugate, indicating complete preservation of CPT during activation and conjugation reactions.

### Example 38

### HA-6-NHCO(CH₂)₂—CO-20-O-CPT Sodium Salt

To a solution of 1.03 g (2.30 mmol) of CPT-20-O-hemisuccinate from Example 34 and 318 mg (2.76 mmol) of N-hydroxysuccinimide in 30 ml of dimethylsulfoxide were added, with stirring under nitrogen at room temperature, 356 μL (2.30 mmol) of diisopropylcarbodiimide. After 16 h, 1.50 g (2.30 mmol) of 6-NH₂-HA TBA salt from Example 20 were added, and stirring was continued for 5 h. 3.0 ml of saturated NaCl solution were then added and stirring was continued for 30 min. The mixture was poured into 120 ml of EtOH while stirring, the resulting slurry was stirred for 10 min and then filtered and washed with EtOH. The solid was suspended in 100 ml of dimethylformamide, slurried for 30 min, filtered and washed once with dimethylformamide and twice with MeOH. After drying on the filter, the solid was dissolved in 100 ml of water and dialysed against water. Then the solution was filtered through a 0.22μ pore size membrane and freeze-dried to give 1.01 g of a white solid. DS in CPT by proton NMR: 25% mol/mol. DS in CPT by HPLC: 13% w/w

### Example 39

### HA-6-NHCO(CH₂)₂—CO-20-O-CPT Sodium Salt

To a solution of 1.03 g (2.30 mmol) of CPT-20-O-hemisuccinate from Example 34 and 400 mg (3.48 mmol) of N-hydroxysuccinimide in 30 ml of dimethylsulfoxide were added, with stirring under nitrogen at room temperature, 356 μL (2.30 mmol) of diisopropylcarbodiimide. After 16 h, 1.50 g (2.30 mmol) of 6-NH₂-HA TBA salt from Example 21 were added, and stirring was continued for 5 h. 3.0 ml of saturated NaCl solution were then added and stirring was continued for 30 min. The mixture was poured into 120 ml of EtOH while stirring, the resulting slurry was stirred for 10 min and then filtered and washed with EtOH. The solid was suspended in 100 ml of dimethylformamide, slurried for 30 min, filtered and washed once with dimethylformamide and twice with MeOH. After drying on the filter, the solid was dissolved in 100 ml of water and dialysed against water. Then the solution was filtered through a 0.22μ pore size membrane and freeze-dried to give 0.99 g of a white solid. DS in CPT by proton NMR: 12% mol/mol. DS in CPT by HPLC: 7% w/w

Alternatively, starting from 1.00 g of 6-NH₂-HA TBA salt from Example 26, 653 mg of a white solid were obtained (DS in CPT 12% mol/mol from ¹H NMR).

### Example 40

### HA-6-NHCO(CH₂)₂—CO-20-O-CPT Sodium Salt

To a solution of 1.03 g (2.30 mmol) of CPT-20-O-hemisuccinate from Example 34 and 400 mg (3.48 mmol) of N-hydroxysuccinimide in 30 ml of dimethylsulfoxide were added, with stirring under nitrogen at room temperature, 356 μL (2.30 mmol) of diisopropylcarbodiimide. After 16 h, 1.50 g (2.30 mmol) of 6-NH₂-HA TBA salt from Example 16 were added, and stirring was continued for 6 h. 3.0 ml of saturated NaCl solution were then added and stirring was continued for 30 min. The mixture was poured into 120 ml of EtOH while stirring, the resulting slurry was stirred for 10 min and then filtered and washed with EtOH. The solid was suspended in 100 ml of dimethylformamide, slurried for 30 min, filtered and washed once with dimethylformamide and twice with MeOH. After drying on the filter, the solid was dissolved in 100 ml of water and dialysed against water. Then the solution was filtered through a 0.22μ pore size membrane and freeze-dried to give 1.26 g of a white solid. DS in CPT by proton NMR: 33% mol/mol.

### Example 41

### HA-6-NHCO(CH₂)₂—CO-20-O-CPT Sodium Salt

To a solution of 867 mg (1.94 mmol) of CPT-20-O-hemisuccinate from Example 34 and 334 mg (2.90 mmol) of N-hydroxysuccinimide in 30 ml of dimethylsulfoxide were added, with stirring under nitrogen at room temperature, 300 μL (1.94 mmol) of diisopropylcarbodiimide. After 16 h, 1.50 g (2.30 mmol) of 6-NH₂-HA TBA salt from Example 22 were added, and stirring was continued for 6 h. 3.0 ml of saturated NaCl solution were then added and stirring was continued for 30 min. The mixture was poured into 120 ml of EtOH while stirring, the resulting slurry was stirred for 10 min and then filtered and washed with EtOH. The solid was suspended in 100 ml of dimethylformamide, slurried for 30 min, filtered and washed once with dimethylformamide and twice with MeOH. After drying on the filter, the solid was dissolved in 100 ml of water and dialysed against water. Then the solution was filtered through a 0.22μ pore size membrane and freeze-dried to give 0.96 g of a white solid. DS in CPT by proton NMR: 3.5% mol/mol. DS in CPT by HPLC: 2% w/w

Alternatively, starting from 1.00 g of 6-NH₂-HA TBA salt from Example 29, 640 mg of a white solid were obtained (DS in CPT 6% mol/mol from ¹H NMR).

### Example 42

### HA-6-NHCO(CH₂)₂—COOH Sodium Salt

To a solution of 1.50 g (2.30 mmol) of 6-NH₂-HA TBA salt from Example 19 in 30 ml of dimethylsulfoxide were added 481 μL (3.45 mmol) of triethylamine and 345 mg (3.45 mmol) of succinic anhydride. After stirring at room temperature overnight, 3.0 ml of saturated NaCl solution were added and stirring was continued for 30 min. The mixture was poured into 150 ml of EtOH while stirring, the resulting slurry was stirred for 10 min and then filtered and washed with EtOH, with 10% water in EtOH, with dimethylformamide and finally with methanol. The solid was dissolved in 100 ml of 0.1N NaOH solution and after five minutes the pH was adjusted to 8 with 3N HCl solution. The solution was ultrafiltered, filtered through a 0.22μ pore size membrane and freeze-dried to give 922 mg of a white solid.

DS in hemisuccinate by proton NMR: 20% mol/mol.

### Example 43

### CPT-20-O—CO—CH₂—NHBoc

To a solution of 3.00 g (17.1 mmol) of Boc-Gly-OH and 1.40 g (11.4 mmol) of 4-dimethylaminopyridine in 100 ml of dichloromethane were added 2.68 ml (17.1 mmol) of diisopropylcarbodiimide and 2.00 g (5.75 mmol) of CPT. After stirring at room temperature overnight, the resulting suspension was diluted with 50 ml of dichloromethane, washed with 0.1N HCl solution, dried over anhydrous sodium sulfate, filtered and evaporated. The residue was crystallized with the minimal amount of methanol, filtered, washed with methanol and dried to give 2.24 g (78%) of a white solid.

### Example 44

### CPT-20-O—CO—CH₂—NH₂-TFA

1.50 g (2.97 mmol) of CPT-20-O—CO—CH₂—NHBoc from Example 43 were dissolved in 100 ml of a 40% solution of trifluoroacetic acid in dichloromethane. After 1 h solvents were removed in a rotary evaporator and the residue was crystallized with diethyl ether, filtered, washed with diethyl ether and dried to give 1.50 g (0.289 mmol, 97%) of a pale yellow solid.

### Example 45

### CPT-20-O—CO—CH₂—NHCO—(CH₂)₂—COOH

To a solution of 174 mg (17.3 mmol) of succinic anhydride, 0.50 ml (2.9 mmol) of DIEA and 4 mg (0.03 mmol) of DMAP in 50 ml of dichloromethane were added, at room temperature under nitrogen, 750 mg (1.44 mmol) of CPT-20-O—CO—CH₂—NH₂-TFA from Example 44. After 18 h, the solution was diluted with 25 ml of dichloromethane, washed with 0.1N HCl solution, washed with saturated NaHCO₃ solution, dried over anhydrous sodium sulfate, filtered and evaporated. The residue was crystallized with the minimal amount of methanol, filtered, washed with methanol and dried to give 655 mg (1.30 mmol, 90%) of a white solid.

### Example 46

### HA-6-NHCO—(CH₂)₂—CONH-Gly-20-O-CPT

To a solution of 250 mg (0.495 mmol) of CPT-20-O—CO—CH₂—NHCO—(CH₂)₂—COOH from Example 45 and 86 mg (0.75 mmol) of N-hydroxysuccinimide in 10 ml of dimethylsulfoxide were added, with stirring under nitrogen at room temperature, 77 μL (0.50 mmol) of diisopropylcarbodiimide. After 16 h, 310 mg (0.50 mmol) of 6—NH₂-HA TBA salt from Example 33 were added, and stirring was continued for 5 h. 1.0 ml of saturated NaCl solution were then added and stirring was continued for 30 min. The mixture was poured into 40 ml of EtOH while stirring, the resulting slurry was stirred for 10 min and then filtered and washed with EtOH. The solid was suspended in 30 ml of dimethylformamide, slurried for 30 min, filtered and washed once with dimethylformamide and twice with MeOH. After drying on the filter, the solid was dissolved in 40 ml of water and dialysed against water. Then the solution was filtered through a 0.22μ pore size membrane and freeze-dried to give 0.22 g (100%) of a white solid.

DS in CPT by proton NMR: 2% mol/mol. DS in CPT by HPLC: 1.5% w/w. Alternatively, starting from 100 mg of 6-NH₂-HA TBA salt from Example 28, 60 mg of a white solid were obtained (DS in CPT 6% mol/mol from ¹H NMR).

### Example 47

### CPT-20-O—CO—(CH₂)₂—CONH-Gly-OH

To a solution of 800 mg (1.79 mmol) of CPT-20-O-hemisuccinate from Example 34, 549 μL (3.94 mmol) of triethylamine and 343 mg (1.79 mmol) of EDC hydrochloride in 80 ml of dichloromethane were added 330 mg (1.96 mmol) of Gly-O-tert-Bu hydrochloride. After stirring at room temperature overnight, the solution was washed with 0.1N HCl solution, dried over anhydrous sodium sulfate, filtered and evaporated in a rotary evaporator. The residue was crystallized with the minimal amount of methanol, filtered, washed with methanol and dried to give a white solid. This solid was dissolved in 25 ml of TFA containing 5% v/v of water. After 1.5 h the solvents were removed in a rotary evaporator and the residue was crystallyzed with methanol. Filtration, washing with methanol and drying gave 601 mg (1.19 mmol, 66%) of an off-white solid.

### Example 48

### CPT-20-O—CO—(CH₂)₂—CONH-Gly-NH-6-HA

To a solution of 250 mg (0.495 mmol) of CPT-20-O—CO—(CH₂)₂—CONH-Gly-OH from Example 47 and 114 mg (0.99 mmol) of N-hydroxysuccinimide in 7 ml of dimethylsulfoxide were added, with stirring under nitrogen at room temperature, 77 μL (0.50 mmol) of diisopropylcarbodiimide. After 16 h, 250 mg (0.403 mmol) of 6—NH₂-HA TBA salt from Example 19 were added, and stirring was continued for 5 h. 0.7 ml of saturated NaCl solution were then added and stirring was continued for 30 min. The mixture was poured into 30 ml of EtOH while stirring, the resulting slurry was stirred for 10 min and then filtered and washed with EtOH. The solid was suspended in 25 ml of dimethylformamide, slurried for 30 min, filtered and washed once with dimethylformamide and twice with MeOH. After drying on the filter, the solid was dissolved in 30 ml of water and dialysed against water. Then the solution was filtered through a 0.22μ pore size membrane and freeze-dried to give 120 mg of a white solid.

DS in CPT by proton NMR: 20% mol/mol. DS in CPT by HPLC: 14.0% w/w. Alternatively, starting from 100 mg of 6-NH₂-HA TBA salt from Example 28, 63 mg of a white solid were obtained (DS in CPT 6% mol/mol from ¹H NMR; DS in CPT by HPLC: 4.82% w/w).

### Example 49

### Taxol-2′-hemisuccinate

A solution of 300 mg (0.351 mmol) of taxol, 42.2 mg (0.422 mmol) of succinic anhydride and 9 mg (0.07 mmol) of DMAP in 10 ml of dry pyridine was stirred at room temperature for 3 h. The solvent was removed in a rotary evaporator and the residue was dissolved in the minimal amount of dichloromethane and charged on a silica gel column. Elution with methanol in ethylacetate (from 0% to 100%) gave 334 mg (100%) of pure taxol-2′-hemisuccinate.

### Example 50

### HA-6-NHCO—(CH₂)₂—CO-2′-O-TXL

To a solution of 334 mg (0.351 mmol) of taxol-2′-hemisuccinate from Example 49 and 115 mg (1.00 mmol) of N-hydroxysuccinimide in 10 ml of dimethylsulfoxide were added, with stirring under nitrogen at room temperature, 62 μL (0.35 mmol) of diisopropylcarbodiimide. After 16 h, 217 mg (0.35 mmol) of 6-NH₂-HA TBA salt from Example 23 were added, and stirring was continued for 7 h. 0.7 ml of saturated NaCl solution were then added and stirring was continued for 30 min. The mixture was poured into 40 ml of EtOH while stirring, the resulting slurry was stirred for 10 min and then filtered and washed with EtOH and MeOH. The solid was dissolved in 25 ml of water and dialysed against water. Then the solution was filtered through a 0.22μ pore size membrane and freeze-dried to give 130 mg of a white solid. DS in TXL by proton NMR: 2% mol/mol. Alternatively, starting from 100 mg of 6-NH₂-HA TBA salt from Example 28, 62 mg of a white solid were obtained (DS in TXL 5% mol/mol from ¹H NMR).

### Example 51

### HA-6-NH-MTX

To a solution of 330 mg (0.726 mmol) of methotrexate and 56 mg (0.484 mmol) of N-hydroxysuccinimide in 6 ml of dimethylsulfoxide were added, with stirring under nitrogen at room temperature, 75 μL (0.484 mmol) of diisopropylcarbodiimide. After 16 h, 300 mg (0.484 mmol) of 6-NH₂-HA TBA salt from Example 21 were added, and stirring was continued for 4.5 h. 1.0 ml of saturated NaCl solution were then added and stirring was continued for 30 min. The mixture was poured into 30 ml of EtOH while stirring, the resulting slurry was stirred for 10 min and then filtered and washed with EtOH and 3×DMF. The solid was dissolved in 20 ml of water and dialysed against water. Then the solution was filtered through a 0.22μ pore size membrane and freeze-dried to give 202 mg of a yellow solid. DS in MTX by proton NMR: 13% mol/mol.

Alternatively, starting from 1.00 g of 6-NH₂-HA TBA salt from Example 25, 706 mg of a yellow solid were obtained (DS in MTX 20% mol/mol from ¹H NMR).

### Example 52

### HA-6-NH-Ibuprofen

To a solution of 110 mg (0.532 mmol) of ibuprofen and 61 mg (0.532 mmol) of N-hydroxysuccinimide in 6 ml of dimethylsulfoxide were added, with stirring under nitrogen at room temperature, 75 μL (0.484 mmol) of diisopropylcarbodiimide. After 16 h, 300 mg (0.484 mmol) of 6-NH₂-HA TBA salt from Example 21 were added, and stirring was continued for 4.5 h. 1.0 ml of saturated NaCl solution were then added and stirring was continued for 30 min. The mixture was poured into 30 ml of EtOH while stirring, the resulting slurry was stirred for 10 min and then filtered and washed with EtOH and 3×DMF. The solid was dissolved in 20 ml of water and dialysed against water. Then the solution was filtered through a 0.22μ pore size membrane and freeze-dried to give 180 mg of a white solid. DS in ibuprofen by proton NMR: 13% mol/mol.

Alternatively, starting from 1.00 g of 6-NH₂-HA TBA salt from Example 25, 690 mg of a white solid were obtained (DS in ibuprofen 20% mol/mol from ¹H NMR).

### Example 53

### Preparation of 6-NH₂-HA Sodium Salt

5.0 g of 6-Cl-HA from Example 8 were dissolved in 100 ml of conc. NH₄OH solution, in a reactor suitable for reactions under pressure. The reactor was sealed and the solution was heated at 80° C. for 22 h, then it was cooled and excess ammonia was removed under vacuum. The solution was treated with 10 ml of saturated sodium chloride and stirred for 30 min. Then it was ultrafiltered and freeze-dried to afford 3.35 g of 6-NH₂-HA sodium salt as an off-white solid (DS 20% mol/mol, determined by ¹³C NMR).

### Example 54

### HA-6-NH-(4-formyl-benzoyl)

To a solution of 600 mg (4.00 mmol) of 4-carboxy-benzaldehyde, 506 mg (4.40 mmol) of N-hydroxysuccinimide and 0.67 ml (4.8 mmol) of triethylamine in 30 ml of DCM, were added 767 mg (4.00 mmol) of EDC hydrochloride. After stirring at room temperature for 4 h, the solution was washed twice with 0.1N HCl solution and twice with water. Then it was dried over anhydrous sodium sulfate and evaporated to dryness to obtain 840 mg (90%) of a white solid. 50 mg of this solid were dissolved in 0.60 ml of DMF and added to a solution of 100 mg of 6-NH₂-HA sodium salt from Example 53 in 5 ml of phosphate buffer (pH=7.2). After stirring for 3 h at room temperature, the resulting suspension was diluted to 15 ml with water, centrifuged to remove solids, dialysed against water and freeze-dried to give 92 mg of a white solid.

DS of 4-formyl-benzoyl groups by proton NMR: 12% mol/mol.

### Example 55

### HA-6-NH-(4-pentylaminomethyl-benzoyl)

To a solution of 82 mg (0.205 mmol) of HA-6-NH-(4-formyl-benzoyl) from Example 54 in 3.7 ml of 0.2M NaHCO₃ solution were added 24 μL (0.205 mmol) of pentylamine and 13 mg (0.205 mmol) of sodium cyanoborohydride. After stirring overnight at room temperature, the suspension was diluted to 11 ml with water, centrifuged to remove solids, dialysed against water and freeze-dried to give 75 mg of a white solid.

DS of acyl groups by proton NMR: 6% mol/mol.

### Example 56

### HA-6-NH—Ac

To a suspension of 80 mg (0.129 mmol) of 6-NH₂-HA TBA salt from Example 19 in 10 ml of acetic anhydride was added dimethylformamide (10 ml) to obtain a solution which was stirred at room temperature for 16 h. 0.5 ml of saturated NaCl solution were then added. After stirring for 30 min the mixture was poured onto EtOH and filtered. The solids were dissolved in 10 ml of 0.1N NaOH solution. After 10 min the solution was neutralized, ultrafiltered and freeze-dried to afford 45 mg of a white solid (DS in acetyl 20% from ¹H NMR).

Alternatively, starting from 200 mg of 6-NH₂-HA TBA salt from Example 26, 124 mg of a white solid were obtained (DS in acetyl 12% mol/mol from ¹H NMR).

### Example 57

### HA-6-NH-picolinoyl

To a solution of 57 mg (0.46 mmol) of picolinic acid in 12 ml of DMSO were added, under nitrogen, 80 mg (0.70 mmol) of NHS and 71 μL (0.46 mmol) of diisopropylcarbodiimide. After stirring for 18 h at room temperature, 300 mg (0.46 mmol) of 6-NH₂-HA TBA salt from Example 21 were added and stirring was continued for 6 h. 1.5 ml of saturated NaCl solution were then added and stirring was continued for 30 min. The mixture was poured into 25 ml of EtOH and then filtered. The solid was washed with EtOH and then dissolved in 20 ml of 0.1N NaOH solution. After stirring for 10 min the solution was neutralized with 0.1N HCl solution, ultrafiltered and freeze-dried to give 180 mg of a white solid (DS in picolinoyl 13% from ¹H NMR).

Alternatively, starting from 200 mg of 6-NH₂-HA TBA salt from Example 26, 117 mg of a white solid were obtained (DS in picolinoyl 12% mol/mol from ¹H NMR).

### Example 58

### Cbz-Gly-6-HN-HA

To a solution of 67 mg (0.323 mmol) of Cbz-Gly-OH and 56 mg (0.484 mmol) of N-hydroxysuccinimide in 4 ml of dimethylsulfoxide were added, with stirring under nitrogen at room temperature, 50 μL (0.323 mmol) of diisopropylcarbodiimide. After 16 h, 200 mg (0.323 mmol) of 6-NH₂-HA TBA salt from Example 21 were added, and stirring was continued for 5 h. 0.40 ml of saturated NaCl solution were then added and stirring was continued for 30 min. The mixture was poured into 15 ml of EtOH while stirring, the resulting slurry was stirred for 10 min and then filtered and washed with EtOH. The solid was dissolved in 15 ml of water and dialysed against water. Then the solution was filtered through a 0.22μ pore size membrane and freeze-dried to give 137 mg of a white solid.

DS in Cbz-Gly by proton NMR: 13% mol/mol.

### Example 59

### H₂N-Gly-6-HN-HA

70 mg of Cbz-Gly-6-HN-HA from Example 58 were dissolved in 1.5 ml of water containing 50 mg of ammonium formate. After purging by several vacuum/nitrogen cycles, the solution was charged with 40 mg of 10% Pd/C (wet) and then stirred at room temperature for 18 h. After dilution to 8 ml with water, the mixture was centrifuged and the solids were discarded. The resulting blackish solution was passed through a short pad of celite, concentrated and freeze-dried to afford 55 mg of an off-white solid.

DS in Gly by proton NMR: 13% mol/mol.

### Example 60

### HA-6-NHCO-Ph

To a solution of 250 mg (0.403 mmol) of 6-NH₂-HA TBA salt from Example 21 and 84 μL (0.604 mmol) of triethylamine in 7 ml of DMSO, were added, with stirring under nitrogen at room temperature, 47 μL (0.403 mmol) of benzoyl chloride. After 3 h the solution was treated with 1 ml of saturated NaCl solution and stirring was continued for 30 min. The mixture was poured into 20 ml of EtOH while stirring, the resulting slurry was stirred for 10 min and then filtered and washed with EtOH. The solid was dissolved in 15 ml of water and dialysed against water. Then the solution was filtered through a 0.22μ pore size membrane and freeze-dried to give 157 mg of a white solid. DS in Bz by proton NMR: 6% mol/mol.

Alternatively, starting from 200 mg of 6-NH₂-HA TBA salt from Example 26, 110 mg of a white solid were obtained (DS in Bz 12% mol/mol from ¹H NMR).

### Example 61

### HA-6-NHCO-(o-amino-phenyl)

To a solution of 250 mg (0.403 mmol) of 6-NH₂-HA TBA salt from Example 17 and 84 μL (0.604 mmol) of triethylamine in 10 ml of DMSO, were added, with stirring under nitrogen at room temperature, 66 mg (0.403 mmol) of isatoic anhydride. After 18 h the solution was treated with 1 ml of saturated NaCl solution and stirring was continued for 30 min. The mixture was poured into 20 ml of EtOH while stirring, the resulting slurry was stirred for 10 min and then filtered and washed with EtOH. The solid was dissolved in 15 ml of 0.1N NaOH solution. After stirring for 10 min the solution was neutralized with 1N HCl solution and dialysed against water. Then the solution was filtered through a 0.22μ pore size membrane and freeze-dried to give 175 mg of a white solid.

DS in antranoyl by proton NMR: 40% mol/mol.

Alternatively, starting from 200 mg of 6-NH₂-HA TBA salt from Example 25, 134 mg of a white solid were obtained (DS in antranoyl 20% mol/mol from ¹H NMR).

### Example 62

### HA-6-NHCO-ⁿPr

To a solution of 37 μL (0.403 mmol) of butyric acid and 70 mg (0.604 mmol) of N-hydroxysuccinimide in 2 ml of dimethylsulfoxide were added, with stirring under nitrogen at room temperature, 62 μL (0.403 mmol) of diisopropylcarbodiimide. After 3 h, 250 mg (0.403 mmol) of 6-NH₂-HA TBA salt from Example 20 were added, and stirring was continued for 16 h. 1.0 ml of saturated NaCl solution were then added and stirring was continued for 30 min. The mixture was poured into 15 ml of EtOH while stirring, the resulting slurry was stirred for 10 min and then filtered and washed with EtOH. The solid was dissolved in 15 ml of 0.1N NaOH solution. After stirring for 10 min the solution was neutralized with 1N HCl solution and dialysed against water. Then the solution was filtered through a 0.22μ pore size membrane and freeze-dried to give 131 mg of a white solid.

DS in butyroyl by proton NMR: 25% mol/mol.

Alternatively, starting from 200 mg of 6-NH₂-HA TBA salt from Example 25, 120 mg of a white solid were obtained (DS in butyroyl 20% mol/mol from ¹H NMR).

### Example 63

### HA-6-NH—(CO)—CH₂—CH(NHCbz)-COOH

To a solution of 300 mg (0.46 mmol) of 6-NH₂-HA TBA salt from Example 21 in 12 ml of DMSO were added, stirring at room temperature under nitrogen, 128 μL (0.92 mmol) of triethylamine, 11 mg (0.09 mmol) of DMAP and 137 mg (0.55 mmol) of N-Carbobenziloxy-L-aspartic anhydride. After 16 h, 1.0 ml of saturated NaCl solution were added and stirring was maintained for 30 min. The mixture was then poured into 25 ml of EtOH and filtered. The solids were dissolved in 5 ml of 0.1N NaOH. After 10 min the solution was neutralized, ultrafiltered and freeze-dried to give 110 mg of a white solid (DS 13% mol/mol in acyl from ¹H NMR).

### Example 64

### 5α-cholestan-3β-ol Hemisuccinate

To a solution of 1.00 g (2.5 mmol) of 5α-cholestan-3β-ol in 50 ml of dichloromethane were added, stirring at 0° C. under nitrogen, 672 mg (3.8 mmol) of mono tert-butyl succinate, 235 mg (1.9 mmol) of DMAP and 958 mg (5.0 mmol) of EDC hydrochloride. After 30 min the mixture was taken to room temperature and stirred for 1 h. The solution was then washed with 10% w/v citric acid solution, saturated NaHCO₃ solution and saturated NaCl solution. Then it was dried over anhydrous sodium sulfate, filtered and evaporated to dryness. The solid was treated with a mixture of 20 ml of trifluoroacetic acid and 1 ml of water and the resulting solution was stirred for 2 h at room temperature. Evaporation to dryness gave 830 mg of a white solid.

### Example 65

### HA-6-NH—CO—(CH₂)₂—CO-3β-O-5α-cholestane

To a solution of 252 mg (0.46 mmol) of 5α-cholestan-3β-ol hemisuccinate in 12 ml of DMSO were added, under nitrogen, 80 mg (0.70 mmol) of NHS and 71 μL (0.46 mmol) of diisopropylcarbodiimide. After stirring for 18 h at room temperature, 300 mg (0.46 mmol) of 6-NH₂-HA TBA salt from Example 21 were added and stirring was continued for 6 h. 1.5 ml of saturated NaCl solution were then added and stirring was continued for 30 min. The mixture was poured into 25 ml of EtOH and then filtered. The solid was washed with EtOH and then dissolved in 20 ml of 0.1N NaOH solution. After stirring for 10 min the solution was neutralized with 0.1N HCl solution, ultrafiltered and freeze-dried to give 197 mg of a white solid (DS in acyl 13% from ¹H NMR).

Alternatively, starting from 200 mg of 6-NH₂-HA TBA salt from Example 28, 124 mg of a white solid were obtained (DS in acyl 6% mol/mol from ¹H NMR).

### Example 66

### HA-6-NH—(N-Cbz-prolinoyl)

To a solution of 114 mg (0.46 mmol) of N-Cbz-L-proline in 12 ml of DMSO were added, under nitrogen, 80 mg (0.70 mmol) of NHS and 71 μL (0.46 mmol) of diisopropylcarbodiimide. After stirring for 18 h at room temperature, 300 mg (0.46 mmol) of 6-NH₂-HA TBA salt from Example 21 were added and stirring was continued for 6 h. 1.5 ml of saturated NaCl solution were then added and stirring was continued for 30 min. The mixture was poured into 25 ml of EtOH and then filtered. The solid was washed with EtOH and then dissolved in 20 ml of 0.1N NaOH solution. After stirring for 10 min the solution was neutralized with 0.1N HCl solution, ultrafiltered and freeze-dried to give 182 mg of a white solid (DS in acyl 13% from ¹H NMR).

Alternatively, starting from 200 mg of 6-NH₂-HA TBA salt from Example 26, 113 mg of a white solid were obtained (DS in acyl 11% mol/mol from ¹H NMR).

### Example 67

### HA-6-NH—(S)—CH(COOH)—(CH₂)₄—NH₂

To a solution of 250 mg (0.403 mmol) of HA-6-O-Ms from Example 14 in 10 ml of DMSO were added, stirring under nitrogen at room temperature, 69 μL (0.403 mmol) of DIEA and 295 mg (2.02 mmol) of L-lysine. The solution was heated to 70° C. and stirred for 18 h, then it was cooled, poured into water, neutralized with diluted HCl solution, treated with 2 ml of saturated NaCl solution, ultrafiltered and freeze-dried to give 140 mg of an off-white solid (DS in lysine 30% mol/mol from ¹H NMR).

### Example 68

### HA-6-O—CO—(CH₂)₂—NHCbz

To a solution of 1.00 g (1.40 mmol) of 6-OMs-HA TBA salt from Example 13 and 937 mg (4.2 mmol) of Cbz-β-Ala in 25 ml of DMSO were added, stirring at room temperature under nitrogen, 236 mg (0.70 mmol) of anhydrous cesium carbonate. The mixture was then heated to 70° C. and stirred for 18 h. Then it was cooled and poured into ice water (100 ml). The pH was adjusted between 6.5 and 7 and 10 ml of saturated NaCl solution were added. The solution was ultrafiltered and freeze dried to afford 550 mg of a white solid.

DS in Cbz-β-Ala 18% mol/mol by proton NMR.

### Example 69

### HA-6-O—CO—(CH₂)₂—NH₂

To a solution of 100 mg of HA-6-O—CO—(CH₂)₂—NHCbz from Example 68 and 120 mg of ammonium formate in 2 ml of water were added, after purging by several vacuum/nitrogen cycles, 20 mg of 10% Pd/C (wet). The mixture was stirred for 18 h, then it was centrifuged and filtered through a short pad of celite. After ultrafiltration and freeze-drying, 80 mg of an off-white solid were obtained. Proton NMR showed complete Cbz removal.

### Example 70

### TFA-H₂N-Phe-Gly-20-O-CPT

To a suspension of 348 mg (1.00 mmol) of CPT in 20 ml of DCM were added, stirring at room temperature under nitrogen, 482 mg (1.5 mmol) of Boc-HN-Phe-Gly-OH, 158 mg (1.3 mmol) of DMAP and 309 μL (2.0 mmol) of DIPC. After 16 h the resulting solution was washed with 0.1N HCl solution and with saturated NaHCO₃ solution, then it was dried over anhydrous sodium sulfate, filtered and evaporated to dryness. The solid residue was recrystallized from MeOH/diethyl ether to obtain 350 mg of a solid. This was dissolved in 20 ml of a 80% solution of TFA in DCM and stirred for 2 h at room temperature. The solvents were removed under reduced pressure and traces of TFA were removed by co-evaporating with small portions of diethyl ether. The residue was crystallized from MeOH/diethyl ether to obtain 320 mg of a solid.

### Example 71

### HOOC—(CH₂)₂CO-Phe-Gly-20-O-CPT

To a solution of 60 mg (0.60 mmol) of succinic anhydride, 171 μL (1.0 mmol) of DIEA and 12 mg (0.1 mmol) of DMAP in 20 ml of DCM was added, stirring at room temperature under nitrogen, 320 mg (0.52 mmol) of TFA-H₂N-Phe-Gly-20-O-CPT from Example 70. After 16 h the solution was diluted with 20 ml of DCM, washed with 0.1N HCl solution and dried over anhydrous sodium sulfate. After filtration and evaporation of the solvent, the residue was crystallized from MeOH/diethyl ether to obtain 150 mg of a pale yellow solid.

### Example 72

### HA-6-NH—CO—(CH₂)₂—CO—HN-Phe-Gly-20-O-CPT

To a solution of 75 mg (0.105 mmol) of HOOC—(CH₂)₂CO-Phe-Gly-20-O-CPT from Example 71 in 1 ml of DMSO were added, stirring at room temperature under nitrogen, 18.2 mg (0.158 mmol) of NHS and 16 μL (0.105 mmol) of DIPC. After 16 h, 71 mg (0.11 mmol) of 6-NH₂-HA TBA salt from Example 21 were added and stirring was continued for 6 h. 0.15 ml of saturated NaCl solution were then added and the mixture was stirred for 30 min. Then 6 ml of EtOH were added under stirring and the mixture was filtered. The solids were washed with DMF and EtOH, then dissolved in 5 ml of water and dialysed against water. Freeze-drying afforded 45 mg of a white solid. DS in CPT by proton NMR: 13% mol/mol.

Alternatively, starting from 200 mg of 6-NH₂-HA TBA salt from Example 26, 128 mg of a white solid were obtained (DS in acyl 12% mol/mol from ¹H NMR).

### Example 73

### TFA-H₂N-Phe-Leu-Gly-20-O-CPT

To a suspension of 348 mg (1.00 mmol) of CPT in 20 ml of DCM were added, stirring at room temperature under nitrogen, 653 mg (1.5 mmol) of Boc-HN-Phe-Leu-Gly-OH, 158 mg (1.3 mmol) of DMAP and 309 μL (2.0 mmol) of DIPC. After 16 h the resulting solution was washed with 0.1N HCl solution and with saturated NaHCO₃ solution, then it was dried over anhydrous sodium sulfate, filtered and evaporated to dryness. The solid residue was recrystallized from MeOH/diethyl ether to obtain 480 mg of a solid. This was dissolved in 5 ml of a 40% solution of TFA in DCM and stirred for 2 h at room temperature. The solvents were removed under reduced pressure and traces of TFA were removed by co-evaporating with small portions of diethyl ether obtaining 492 mg of a solid residue.

### Example 74

### HOOC—(CH₂)₂CO-Phe-Leu-Gly-20-O-CPT

To a solution of 492 mg (0.63 mmol) of TFA-H₂N-Phe-Leu-Gly-20-O-CPT from Example 73 in 10 ml of DMF were added, stirring at 0° C. under nitrogen, 109 mg (0.63 mmol) of mono tert-butyl succinate, 85 mg (0.63 mmol) of HOBt, 216 μL (1.26 mmol) of DIEA and 146 mg (0.76 mmol) of EDC hydrochloride. The reaction mixture was then allowed to reach room temperature overnight. The solvent was removed under reduced pressure and the residue partitioned between EtOAc and water. The aqueous phase was extracted with EtOAc. The combined organic phases were washed with 10% citric acid, saturated NaHCO₃ solution and brine, then they were dried over anhydrous sodium sulfate, filtered and evaporated to dryness. The residue was dissolved in 5 ml of a 40% solution of TFA in DCM. After 2 h solvents were removed under reduced pressure and traces of TFA were removed by co-evaporating with small portions of diethyl ether obtaining 490 mg of a solid.

### Example 75

### HA-6-NH—CO—(CH₂)₂—CO—HN-Phe-Leu-Gly-20-O-CPT

To a solution of 490 mg (0.63 mmol) of HOOC—(CH₂)₂CO-Phe-Leu-Gly-20-O-CPT from Example 74 in 20 ml of DMSO were added, stirring at room temperature under nitrogen, 108 mg (0.90 mmol) of NHS, 154 μL (0.90 mmol) of DIEA and 132 mg (0.70 mmol) of EDC hydrochloride. After 16 h, 390 mg (0.63 mmol) of 6-NH₂-HA TBA salt from Example 21 were added and stirring was continued for 6 h. 2 ml of saturated NaCl solution were then added and the mixture was stirred for 30 min. Then 80 ml of EtOH were added under stirring and the mixture was filtered. The solids were washed with DMF and EtOH, then dissolved in 15 ml of water and dialysed against water. Freeze-drying afforded 194 mg of a white solid. DS in CPT by proton NMR: 13% mol/mol.

Alternatively, starting from 300 mg of 6-NH₂-HA TBA salt from Example 26, 222 mg of a white solid were obtained (DS in acyl 12% mol/mol from ¹H NMR).

### Example 76

### CPT-20-O—CO—(CH₂)₂—CO—HN-Gly-Phe-6-HN-HA

To a solution of 280 mg (1.0 mmol) of TFA-H₂N-Gly-Phe-O^{t}Bu in 20 ml of DMF were added, stirring at 0° C. under nitrogen, 450 mg (1.0 mmol) of CPT hemisuccinate from Example 34, 135 mg (1.0 mmol) of HOBt, 342 μL (2.0 mmol) of DIEA and 230 mg (1.2 mmol) of EDC hydrochloride. The reaction mixture was then allowed to reach room temperature overnight. The solvent was removed under reduced pressure and the residue partitioned between EtOAc and water. The aqueous phase was extracted with EtOAc. The combined organic phases were washed with 10% citric acid, saturated NaHCO₃ solution and brine, then they were dried over anhydrous sodium sulfate, filtered and evaporated to dryness. The residue was dissolved in 8 ml of a 40% solution of TFA in DCM. After 2 h solvents were removed under reduced pressure and traces of TFA were removed by co-evaporating with small portions of diethyl ether obtaining a solid. To this solid, dissolved in 20 ml of DMSO, were added, stirring at room temperature under nitrogen, 172 mg (1.5 mmol) of NHS, 171 μL (0.90 mmol) of DIEA and 154 μL (1.0 mmol) of DIPC. After 16 h, 620 mg (0.63 mmol) of 6-NH₂-HA TBA salt from Example 19 were added and stirring was continued for 6 h. 2 ml of saturated NaCl solution were then added and the mixture was stirred for 30 min. Then 100 ml of EtOH were added under stirring and the mixture was filtered. The solids were washed with DMF and EtOH, then dissolved in 20 ml of water and dialysed against water. Freeze-drying afforded 406 mg of a white solid. DS in CPT by proton NMR: 20% mol/mol.

Alternatively, starting from 300 mg of 6-NH₂-HA TBA salt from Example 26, 205 mg of a white solid were obtained (DS in acyl 12% mol/mol from ¹H NMR).

### Example 77

### Antiproliferative Activity

Antiproliferative activity of the DDS was determined on three lines of carcinoma (HT29: colon rectal carcinoma, H460: lung carcinoma, H460M2: lung metastatic carcinoma which are CPT sensitive. Cells were incubated in 96-well plates for 5 days in complete RPMI1640 Medium (Sigma Chemical Co.) supplemented with 10% FBS (Hyclone Europe), 2 mM L-glutamine (Hyclone Europe), and 100 U/ml penicillin G and 100 ug/ml streptomycin (Sigma Chemical Co.) at 370, and in controlled atmosphere (5% CO₂), with irinotecan and DDSs of examples 38, 39, 41; the DDS were tested at doses equimolar with those of the reference, in the range 3-30 nM and 0.1-30 μM.

Cytotoxicity was determined on day 6 (after 5 days treatments) by the MTT test, by measuring cell viability as the cell metabolic capacity to transform the tetrazolium salt of MTT in the blue formazan, by mitochondrial dehydrogenases; the blue colour is read at 570 nm with a spectrophotometer. The following DDSs were tested.

| Example | % SUBSTITUTION |
|---|---|
| Ex 41 | 2% |
| Ex 39 | 7% |
| Ex 38 | 13% |

Antitumour effect of the DDS of the invention on various tumour cells is reported in the table A; the effect of these DDSs has been compared to that of irinotecan. This reference is the CPT analogs that is currently used in therapy; CPT is insoluble in aqueous solution and therefore it cannot be used in therapy and for both reasons it is not a suitable reference.

Table A shows the values of the concentration (IC₅₀, μM) of the DDS and of irinotecan necessary to reduce the cell growth of various tumours lines to 50% of the growth of the control.

TABLE A

| Cell line | Irinotecan | Example 41 | Example 39 | Example 38 |
|---|---|---|---|---|
| HT29 | 250.00 | 29.33 | 29.02 | 37.85 |
| (colon rectal | | | | |
| carcinoma) | | | | |
| H460 | 440.00 | 14.11 | 9.52 | 32.33 |
| (lung carcinoma) | | | | |
| H460-M2 | 460.00 | 23.30 | 33.11 | 47.50 |
| (lung | | | | |
| metastatic | | | | |
| carcinoma) | | | | |

These data show that the present DDS have a very high antiproliferative activity. 6-NH₂-HA and HA-6-NH-succinate, which are intermediate compounds used in the preparation of the final DDS, have also been tested under the same conditions and the test shows that they are not cytotoxic.

### Example 78

### HA-6-NH-naproxen

To a solution of 93 mg (0.403 mmol) of naproxen and 70 mg (0.604 mmol) of N-hydroxysuccinimide in 2 ml of dimethylsulfoxide were added, with stirring under nitrogen at room temperature, 62 μL (0.403 mmol) of diisopropylcarbodiimide. After 3 h, 250 mg (0.403 mmol) of HA-6-NH₂ TBA salt made as in Example 19 were added, and stirring was continued for 16 h. 1.0 ml of saturated NaCl solution were then added and stirring was continued for 30 min. The mixture was poured into 15 ml of EtOH while stirring, the resulting slurry was stirred for 10 min and then filtered and washed with EtOH. The solid was dissolved in 15 ml of 0.1N NaOH solution. After stirring for 10 min the solution was neutralized with 1N HCl solution and dialysed against water. Then the solution was filtered through a 0.22μ pore size membrane and freeze-dried to give 128 mg of a white solid. DS in naproxen by proton NMR: 20% mol/mol.

Alternatively, starting from 200 mg of HA-6-NH₂ TBA salt made as in Example 26, 125 mg of a white solid were obtained (DS in naproxen 13% mol/mol from ¹H NMR).

### Example 79

### HA-6-NH-lisinopril

To a solution of 178 mg (0.403 mmol) of lisinopril and 70 mg (0.604 mmol) of N-hydroxysuccinimide in 2 ml of dimethylsulfoxide were added, with stirring under nitrogen at room temperature, 62 μL (0.403 mmol) of diisopropylcarbodiimide. After 3 h, 250 mg (0.403 mmol) of HA-6-NH₂ TBA salt made as in Example 19 were added, and stirring was continued for 16 h. 1.0 ml of saturated NaCl solution were then added and stirring was continued for 30 min. The mixture was poured into 15 ml of EtOH while stirring, the resulting slurry was stirred for 10 min and then filtered and washed with EtOH. The solid was dissolved in 15 ml of 0.1N NaOH solution. After stirring for 10 min the solution was neutralized with 1N HCl solution and dialysed against water. Then the solution was filtered through a 0.22μ pore size membrane and freeze-dried to give 140 mg of a white solid. DS in lisinopril by proton NMR: 18% mol/mol.

Alternatively, starting from 200 mg of HA-6-NH₂ TBA salt made as in Example 26, 136 mg of a white solid were obtained (DS in lisinopril 13% mol/mol from ¹H NMR).

### Example 80

### HA-6-NH-nalidixate

To a solution of 94 mg (0.403 mmol) of nalidixic acid and 70 mg (0.604 mmol) of N-hydroxysuccinimide in 2 ml of dimethylsulfoxide were added, with stirring under nitrogen at room temperature, 62 μL (0.403 mmol) of diisopropylcarbodiimide. After 3 h, 250 mg (0.403 mmol) of HA-6-NH₂ TBA salt made as in Example 19 were added, and stirring was continued for 16 h. 1.0 ml of saturated NaCl solution were then added and stirring was continued for 30 min. The mixture was poured into 15 ml of EtOH while stirring, the resulting slurry was stirred for 10 min and then filtered and washed with EtOH. The solid was dissolved in 15 ml of 0.1N NaOH solution. After stirring for 10 min the solution was neutralized with 1N HCl solution and dialysed against water. Then the solution was filtered through a 0.22μ pore size membrane and freeze-dried to give 132 mg of a white solid. DS in nalidixate by proton NMR: 19% mol/mol.

Alternatively, starting from 200 mg of HA-6-NH₂ TBA salt made as in Example 26, 127 mg of a white solid were obtained (DS in nalidixate 12% mol/mol from ¹H NMR).

### Example 81

### HA-6-NH-penicillin G

To a mixture of 144 mg (0.403 mmol) of penicillin G sodium salt and 70 mg (0.604 mmol) of N-hydroxysuccinimide in 2 ml of dimethylsulfoxide were added, with stirring under nitrogen at room temperature, 62 μL (0.403 mmol) of diisopropylcarbodiimide. After 3 h, 250 mg (0.403 mmol) of HA-6-NH₂ TBA salt made as in Example 19 were added, and stirring was continued for 16 h. 1.0 ml of saturated NaCl solution were then added and stirring was continued for 30 min. The mixture was poured into 15 ml of EtOH while stirring, the resulting slurry was stirred for 10 min and then filtered and washed with EtOH. The solid was dissolved in 15 ml of 0.1N NaOH solution. After stirring for 10 min the solution was neutralized with 1N HCl solution and dialysed against water. Then the solution was filtered through a 0.22μ pore size membrane and freeze-dried to give 126 mg of a white solid. DS in penicillin G by proton NMR: 16% mol/mol.

Alternatively, starting from 200 mg of HA-6-NH₂ TBA salt made as in Example 26, 119 mg of a white solid were obtained (DS in penicillin G 11% mol/mol from ¹H NMR).

### Example 82

### HA-6-NH-cefazolin

To a mixture of 192 mg (0.403 mmol) of cefazolin sodium salt and 70 mg (0.604 mmol) of N-hydroxysuccinimide in 2 ml of dimethylsulfoxide were added, with stirring under nitrogen at room temperature, 62 μL (0.403 mmol) of diisopropylcarbodiimide. After 3 h, 250 mg (0.403 mmol) of HA-6-NH₂ TBA salt made as in Example 19 were added, and stirring was continued for 16 h. 1.0 ml of saturated NaCl solution were then added and stirring was continued for 30 min. The mixture was poured into 15 ml of EtOH while stirring, the resulting slurry was stirred for 10 min and then filtered and washed with EtOH. The solid was dissolved in 15 ml of 0.1N NaOH solution. After stirring for 10 min the solution was neutralized with 1N HCl solution and dialysed against water. Then the solution was filtered through a 0.22μ pore size membrane and freeze-dried to give 129 mg of a white solid.

DS in cefazolin by proton NMR: 17% mol/mol.

Alternatively, starting from 200 mg of HA-6-NH₂ TBA salt made as in Example 26, 121 mg of a white solid were obtained (DS in cefazolin 10% mol/mol from ¹H NMR).

### Example 83

### HA-6-NH-Phe-Leu-Gly-Cbz

To a solution of 152 mg (0.323 mmol) of Cbz-Gly-Leu-Phe-OH and 56 mg (0.484 mmol) of N-hydroxysuccinimide in 4 mL of dimethylsulfoxide were added, with stirring under nitrogen at room temperature, 50 μL (0.323 mmol) of diisopropylcarbodiimide. After 16 h, 200 mg (0.323 mmol) of 6-NH₂-HA TBA salt from Example 21 were added, and stirring was continued for 5 h. 0.40 ml of saturated NaCl solution were then added and stirring was continued for 30 min. The mixture was poured into 15 ml of EtOH while stirring, the resulting slurry was stirred for 10 min and then filtered and washed with EtOH. The solid was dissolved in 15 ml of water and dialysed against water. Then the solution was filtered through a 0.22μ pore size membrane and freeze-dried to give 145 mg of a white solid. DS in Phe-Leu-Gly-Cbz by proton NMR: 13% mol/mol.

### Example 84

### HA-6-NH-Phe-Leu-Gly-NH₂

140 mg (0.304 mmol) of HA-6-HN-Phe-Leu-Gly-Cbz from Example 83 were dissolved in 3 mL of water containing 100 mg (1.6 mmol) of ammonium formate. After purging by several vacuum/nitrogen cycles, the solution was charged with 80 mg of 10% Pd/C (wet) and then stirred at room temperature for 18 h. After dilution to 16 mL with water, the mixture was centrifuged and the solids were discarded. The resulting blackish solution was passed through a short pad of celite, concentrated and freeze-dried to afford 113 mg of an off-white solid, which was converted in the corresponding TBA salt by ion exchange on TBA-activated Amberlite. DS in Phe-Leu-Gly by proton NMR: 13% mol/mol.

### Example 85

### HA-6-NH-Phe-Leu-Gly-NH-MTX-OH

To a solution of 100 mg (0.220 mmol) of MTX and 38 mg (0.330 mmol) of NHS in 5 mL of anhydrous DMSO were added, with stirring under nitrogen at room temperature, 35 □L (0.220 mmol) of diisopropylcarbodiimide. After 16 h, 150 mg (0.226 mmol) of HA-6-NH-Phe-Leu-Gly-NH₂ TBA salt prepared in the Example 84 were added, and stirring continued for 5 h. 0.50 mL of saturated NaCl solution were then added and stirring was continued for 30 min. The mixture was poured into 20 mL of EtOH while stirring, the resulting slurry was stirred for 10 min and then filtered and washed with EtOH. The solid was dissolved in 15 mL of water and dialysed against water. Then the solution was filtered through a 0.22μ pore size membrane and freeze-dried to give 110 mg of a yellowish solid. DS in MTX by proton NMR: 13% mol/mol.

### Example 86

### HA-6-NH-Phe-Gly-Cbz

To a solution of 114 mg (0.323 mmol) of Cbz-Gly-Phe-OH and 56 mg (0.484 mmol) of N-hydroxysuccinimide in 4 mL of dimethylsulfoxide were added, with stirring under nitrogen at room temperature, 50 μL (0.323 mmol) of diisopropylcarbodiimide. After 16 h, 200 mg (0.323 mmol) of 6-NH₂-HA TBA salt from Example 21 were added, and stirring was continued for 5 h. 0.40 ml of saturated NaCl solution were then added and stirring was continued for 30 min. The mixture was poured into 15 ml of EtOH while stirring, the resulting slurry was stirred for 10 min and then filtered and washed with EtOH. The solid was dissolved in 15 ml of water and dialysed against water. Then the solution was filtered through a 0.22μ pore size membrane and freeze-dried to give 135 mg of a white solid. DS in Phe-Gly-Cbz by proton NMR: 13% mol/mol.

### Example 87

### HA-6-NH-Phe-Gly-NH₂

130 mg (0.30 mmol) of HA-6-HN-Phe-Gly-Cbz from Example 86 were dissolved in 3 mL of water containing 95 mg (1.5 mmol) of ammonium formate. After purging by several vacuum/nitrogen cycles, the solution was charged with 80 mg of 10% Pd/C (wet) and then stirred at room temperature for 18 h. After dilution to 16 mL with water, the mixture was centrifuged and the solids were discarded. The resulting blackish solution was passed through a short pad of celite, concentrated and freeze-dried to afford 115 mg of an off-white solid, which was converted in the corresponding TBA salt by ion exchange on TBA-activated Amberlite. DS in Phe-Gly by proton NMR: 13% mol/mol.

### Example 88

### HA-6-NH-Phe-Gly-NH-MTX-OH

To a solution of 100 mg (0.220 mmol) of MTX and 38 mg (0.330 mmol) of NHS in 5 mL of anhydrous DMSO were added, with stirring under nitrogen at room temperature, 35 μL (0.220 mmol) of diisopropylcarbodiimide. After 16 h, 150 mg (0.231 mmol) of HA-6-NH-Phe-Gly-NH₂ TBA salt prepared in the Example 87 were added, and stirring continued for 5 h. 0.50 mL of saturated NaCl solution were then added and stirring was continued for 30 min. The mixture was poured into 20 mL of EtOH while stirring, the resulting slurry was stirred for 10 min and then filtered and washed with EtOH. The solid was dissolved in 15 mL of water and dialysed against water. Then the solution was filtered through a 0.22μ pore size membrane and freeze-dried to give 105 mg of a yellowish solid.

DS in MTX by proton NMR: 13% mol/mol.

1. A drug delivery system comprising a 6-amino-hyaluronic acid derivative, wherein the amino group is at the C₆ position of the N-acetyl-D-glucosamine residue of the hyaluronic acid derivative, and a therapeutic agent, wherein the agent is covalently bonded to the derivative by an amidic linkage, directly or via a linker, at the C₆ position of the N-acetyl-D-glucosamine residue of said 6-amino hyaluronic acid derivative.

2. The DDS according to claim 1 where the therapeutic agent contains at least one carboxylic group or at least one amino group or at least one hydroxyl group.

3. The DDS according to claim 2 where the therapeutic agent contains at least one carboxylic group and the amidic linkage between the agent and hyaluronic acid is direct.

4. The DDS according to claim 1 where the therapeutic agent contains at least one amino group or at least one hydroxyl group and the amidic linkage between the agent and hyaluronic acid is via a linker.

5. The DDS according to claim 1 wherein the therapeutic agent is selected from the group consisting of analgesic, antihypertensive, anestetic, diuretic, bronchodilator, calcium channel blocker, cholinergic, CNS agent, estrogen, immunomodulator, immunosuppressant, lipotropic, anxiolytic, antiulcerative, antiarrhytmic, antianginal, antibiotic, anti-inflammatory, antiviral, thrombolitic, vasodilator, antipyretic, antidepressant, antipsychotic, antitumour, mucolytic, narcotic antagonist, hormones, anticonvulsant, antihistaminic, antifungal, and antipsoriatic agents, antiproliferative agents, and antibiotics.

6. The DDS according to claim 5 wherein the therapeutic agent is an anti-inflammatory, antibiotic, or antitumour agent.

7. The DDS according to claim 1 wherein the therapeutic agent is camptothecin, ibuprofen, methotrexate, taxol, cefazolin, naproxen, lisinopril, penicillinG, nalidixic acid, or cholestane, and derivatives thereof.

8. The DDS according to claim 1 wherein the linker is selected from linear or branched, aliphatic, aromatic or araliphatic C₂-C₂₀-dicarboxylic acids, aminoacids, or peptides; linear or branched, aliphatic, aromatic, or araliphatic C₂-C₂₀ dicarboxylic acids linked to aminoacids; or linear or branched aliphatic, aromatic or araliphatic C₂-C₂₀ dicarboxylic acids linked to peptides; each optionally substituted with amino or thiol groups.

9. The DDS according to claim 8 wherein the linker is succinic acid, succinic acid linked to an aminoacid, or succinic acid linked to a peptide.

10. The DDS according to claim 1 wherein the secondary hydroxyl groups of the hyaluronic acid are derivatised to form a group selected from: —OR, —OCOR, —SO₂H, —OPO₃H₂, —O—CO—(CH₂)ₙ—COOH, or —O—(CH₂)ₙ—OCOR, wherein n is 1-4 and R is C₁-C₁₀ alkyl, —NH₂, or —NHCOCH₃.

11. The DDS according to claim 1 wherein the carboxylic group of hyaluronic acid is in the free acid form or is salified with alkaline metals or with earth-alkaline metals or with transition metals.

12. (canceled)

13. A pharmaceutical composition comprising the DDS claim 1 in admixture with pharmaceutically acceptable excipients and/or diluents.

14. The pharmaceutical composition of claim 13 in injectable form.

15. Process for the preparation of the DDS claim 1, comprising forming an amide linkage between 6-aminohyaluronic acid and a —COOH containing therapeutic agent or linker and, when the linker is used, further including the step of linking the therapeutic agent to the linker.

16. Process according to claim 15, wherein said 6-aminohyaluronic acid is obtained from hyaluronic acid, by substituting the hydroxyl group present at the C₆ position in the N-acetyl-D-glucosamine units with an amino group.

17. Process according to claim 16, wherein said substitution is performed by activating the C₆ position with a suitable leaving group, followed by treating with concentrated ammonia, or with sodium azide and a reducing agent.

18. Process according to claim 17, wherein the leaving group is selected from sulfonate, phosphonate (triphenylphoshonate), cyanide, nitrite, halogen, chloro, sulphate, halogensulfate, nitrate, halogensulfite, chlorosulfite.

19. Process according to claim 17 wherein the reagent used for activating the C₆ position is an alkyl- or aryl-sulfonyl halide, and the activation is performed in presence of an organic or inorganic base.

20. Process according to claim 19 wherein the reagent used for activating the C₆ position is methylsulfonyl chloride or toluene-p-sulfonyl chloride and the organic base is diisopropylethylamine or triethylamine.
